(19) United States
(12) Patent Application Publication
(10) Pub. No.: US 2017/0159040 A1
(43) Pub. Date: Jun. 8, 2017

(54) HIGH-EFFICIENCY HYBRID CAPTURE COMPOSITIONS, AND METHODS

(71) Applicants: Justin Lock, San Francisco, CA (US); Hoai Nguyen, San Jose, CA (US); Daniel DeSloover, Millbrae, CA (US)

(72) Inventors: Justin Lock, San Francisco, CA (US); Hoai Nguyen, San Jose, CA (US); Daniel DeSloover, Millbrae, CA (US)

(21) Appl. No.: 15/369,623

(22) Filed: Dec. 5, 2016

Related U.S. Application Data
(60) Provisional application No. 62/263,543, filed on Dec. 4, 2015.
(60) Provisional application No. 62/266,457, filed on Dec. 11, 2015.
(60) Provisional application No. 62/373,887, filed on Aug. 11, 2016.

(51) Int. Cl. C12N 15/10; C12Q 1/68

(52) U.S. Cl. CPC C12N 15/1006; C12Q 1/6874; C12Q 1/6855

(57) ABSTRACT

Methods, and compositions are provided for high-efficiency hybrid capture.

### CROSS-REFERENCE TO RELATED APPLICATIONS

The present application claims priority to U.S. Provisional Application No. 62/263,543, filed Dec. 4, 2015; U.S. Provisional Application No. 62/266,457, filed Dec. 11, 2015; and U.S. Provisional Application No. 62/373,887, filed Aug. 11, 2016. The entire disclosures of each of these applications are incorporated herein by reference in their entireties for all purposes.

### BACKGROUND OF THE INVENTION

Sample preparation for high-throughput nucleic acid sequencing may involve an enrichment step that increases the ratio of target nucleic acids to non-target nucleic acids in a sample. Such enrichment steps can take advantage of a number of different physico-chemical attributes of the target and non-target nucleic acids. See, Mamanova et al., Nat. Methods, 7:111-118 (2010). For example, target nucleic acids having known sequence attributes can be enriched by selecting from a sample nucleic acid fragments having the target sequences. In particular, elevated temperature (e.g., 65° C.) hybridization of target nucleic acids to labeled oligonucleotides (known as bait oligonucleotides) can allow for enrichment of a set of nucleic acids having the target sequences (i.e., target nucleic acids), a process generally referred to as “hybrid capture.” In one approach, hybrid-capture enrichment methods can use RNA bait oligonucleotides, which form RNA:DNA hybrids with target nucleic acids.

Hybrid capture is highly parallelizable as different samples can, e.g., be enriched via hybridization in adjacent wells, tubes, or reaction chambers of an array. Bait oligonucleotides that are hybridized to target nucleic acids can be immobilized (e.g., by binding of label to a functionalized solid surface), washed, and harvested. Consequently, hybrid capture methods are well-suited to high-throughput sequencing work flows that require highly parallelized sample preparation. For high-throughput sequencing sample preparation, the specificity of the hybridization reaction between bait oligonucleotides and sample nucleic acids can be enhanced by including blocking nucleic acid such as CₒT−1 DNA and/or sequence specific blocking oligonucleotides.

However, typical hybrid capture methods known in the art can require very long hybridization times to reach equilibrium and/or achieve efficient capture and enrichment of target nucleic acids. Moreover, although hybrid capture methods known in the art do enrich samples for target nucleic acids, there still remains a significant level of undesirable non-target nucleic acid contamination. Non-target contamination can, inter alia, reduce the probability of detecting rare mutations in enriched nucleic acid samples by high-throughput sequencing. Furthermore, a significant fraction of target nucleic acids can be lost during hybridization, washing, harvesting, or during processing steps upstream (e.g., adaptor ligation) or downstream (e.g., flow cell immobilization) of the hybridization step. Thus, there remains a need in the art for methods, compositions, instrumentation, and systems for hybrid capture enrichment methods, that can reduce hybridization time or sample loss, provide improved efficiency of target enrichment, or a combination thereof. Certain embodiments of the present invention address one or more of these needs.

### BRIEF SUMMARY OF THE INVENTION

The invention relates to improved methods for hybrid capture. In particular, aspects of the invention are directed to hybrid capture of genomic or cDNA libraries used for high-throughput sequencing. In hybrid capture, target nucleic acid molecules in a sample are enriched by hybridizing the target to a mixture of complementary bait oligonucleotides to form target: bait hybrids. The hybrids can be recovered from the sample (e.g., by immobilizing the hybrids to a solid surface) and the target nucleic acids can be eluted from the hybrids to produce a sample enriched for the target nucleic acids.

In one aspect, the present invention provides an aqueous reaction mixture for enrichment of target nucleic acid molecules from a nucleic acid sample comprising a plurality of target nucleic acid molecules and a plurality of non-target nucleic acid molecules, the aqueous reaction mixture comprising: a) a plurality of structurally distinct bait oligonucleotides, wherein the bait oligonucleotides comprise sequences complementary to the plurality of target nucleic acid molecules; b) the plurality of target nucleic acids; c) the plurality of non-target nucleic acids; and d) water, wherein the concentration of bait oligonucleotides in the reaction mixture is at least 0.75 pmol/μL. In some embodiments, the concentration of bait oligonucleotides is from about 1 pmol/μL to about 2 pmol/μL.

In another aspect, the present invention provides a hybrid capture method for enrichment of target nucleic acid molecules from a nucleic acid sample containing target nucleic acid molecules and non-target nucleic acid molecules, the method comprising: i) forming an aqueous reaction mixture described herein; ii) incubating the aqueous reaction mixture at a hybridization temperature for at least about 1 minute and less than about 1 hour to thereby hybridize at least a portion of the bait oligonucleotides to at least a portion of the target nucleic acid molecules; iii) immobilizing at least a portion of the bait oligonucleotides on one or more solid surfaces, thereby producing immobilized target nucleic acid molecule-bait oligonucleotide complexes; iv) separating at least a portion of the non-target nucleic acid molecules from the immobilized target nucleic acid molecule-bait oligonucleotide complexes; and v) recovering target nucleic acid molecules from the one or more solid surfaces, or amplification products thereof, thereby providing a polynucleotide mixture enriched, at least 250-fold for target nucleic acid molecules or baited region relative to the nucleic acid sample.

In another aspect, the present invention provides a hybrid capture method for enrichment of target nucleic acid molecules from a nucleic acid sample containing target nucleic acid molecules and non-target nucleic acid molecules, the method comprising: (i) forming an aqueous reaction mixture described herein, ii) incubating the aqueous reaction mixture at a hybridization temperature (e.g., about 65° C.) for at least about 10 minutes to thereby hybridize at least a portion of the bait oligonucleotides to at least a portion of the target nucleic acid molecules; and, iii) immobilizing at least a portion of the bait oligonucleotides on one or more solid surfaces (e.g., before, after, or during the incubating the aqueous reaction mixture at the hybridization temperature), thereby producing immobilized target nucleic acid molecule-bait oligonucleotide complexes; iv) separating at least a portion of the non-target nucleic acid molecules from the immobilized target nucleic acid molecule-bait oligonucleotide complexes; v) recovering target nucleic acid molecules from the one or more solid surfaces, or amplification products thereof, thereby forming a polynucleotide mixture of target and non-target nucleic acid molecules enriched by target sequences relative to the nucleic acid sample. In some embodiments the method additionally comprises sequencing at least a portion of the target nucleic acids in the enriched polynucleotide mixture. In some embodiments, the hybridization temperature is about 65° C.

In some embodiments, the total concentration of target and non-target nucleic acid molecules in the aqueous reaction mixture is at least about 50 ng/μL. In some embodiments, the total concentration of target and non-target nucleic acid molecules is from about 150 ng/μL to about 300 ng/μL. In some embodiments, the total concentration of target and non-target nucleic acids in the aqueous reaction mixture is 250 ng/μL. In some embodiments, the total concentration of target and non-target nucleic acids in the aqueous reaction mixture is from about 100 ng/μL to about 2,500 ng/μL, or from about 100 ng/μL to about 1,500 ng/μL. In some embodiments, the total concentration of target and non-target nucleic acids in the aqueous reaction mixture is from about 200 ng/μL to about 1,500 ng/μL. In some embodiments, the total concentration of target and non-target nucleic acids in the aqueous reaction mixture is from about 500 ng/μL to about 1,500 ng/μL. In some embodiments, the total concentration of target and non-target nucleic acids in the aqueous reaction mixture is from about 700 ng/μL to about 1,500 ng/μL. In some embodiments, the total concentration of target and non-target nucleic acids in the aqueous reaction mixture is from about 750 ng/μL to about 1,500 ng/μL. In some embodiments, the total concentration of target and non-target nucleic acids in the aqueous reaction mixture is from about 800 ng/μL to about 1,500 ng/μL. In some embodiments, the total concentration of target and non-target nucleic acids in the aqueous reaction mixture is about 500 ng/μL; about 600 ng/μL; about 700 ng/μL; about 800 ng/μL; about 900 ng/μL; about 1,000 ng/μL; or about 1,200 ng/μL.

In some embodiments, the aqueous reaction mixture has a volume of less than about 10 μL, less than about 7 μL, less than about 5 μL, less than about 4 μL, or less than about 3 μL. In some embodiments, the aqueous reaction mixture has a volume of from about 1 μL to 5 μL (e.g., from about 1 μL to 4 μL, or from 1 μL to 3 μL). In some embodiments, the aqueous reaction mixture has a volume of about 2 μL.

As is well known in the sequencing arts, high-throughput sequencing may be carried out using libraries, such as genomic DNA and cDNA libraries, prepared by adding synthetic adaptor sequences to the target DNA molecules. Adaptor sequences may be added to genomic or cDNA by ligating nucleic acid adaptors to, e.g., one or both ends, of sample DNA. Alternatively, adaptor sequences may be added by PCR or other amplification methods. As yet another alternative, adapter sequences may be added by tagmentation (see, e.g., U.S. Pat. No. 9,238,671). Commonly used adaptor sequences include Illumina's P5 and P7 adaptor sequences. A library comprising adaptor(s) associated with sample nucleic acids may be referred to as “adaptor ligated nucleic acid fragments” even if adaptor sequences are added by a method other than ligation.

In some embodiments, the target nucleic acid molecules and non-target nucleic acid molecules consist of a library of adaptor ligated nucleic acid fragments. In some embodiments, the library of adaptor ligated nucleic acid fragments is a library of adaptor ligated genomic DNA fragments. In some embodiments, the library of adaptor ligated nucleic acid fragments is a library of adaptor ligated DNA fragments from a human subject's gut microbiome. In some embodiments, the target nucleic acid molecules and non-target nucleic acid molecules consist of multiple (i.e., two or more) libraries of adaptor ligated nucleic acid fragments. For example, the methods of the invention may be carried out using a mixture of distinguishable libraries such as libraries from more than one individual or more than one cell. Optionally, barcodes or other methods may be used to distinguish libraries with different source DNA. As used herein, reference to “a library” may refer to one library from one source (e.g., an individual) or to libraries from more than one source, unless otherwise indicated.

In some embodiments, the aqueous reaction mixture further comprises blocking nucleic acids. Blocking nucleic acids may be used to hybridize to specific sequences (e.g., adapter sequences on a designated strand or a complement thereof on an opposite other stand) to reduce or eliminate cross-hybridization between different library fragments. In one approach the blocking nucleic acids are oligonucleotides, wherein the blocking oligonucleotides are complementary to one or more adaptors of the adaptor ligated nucleic acid fragments (e.g., complementary to or having the sequence of the Illumina P5 and P7 adaptor sequences). In some embodiments, the aqueous reaction mixture comprises a first blocking oligonucleotide that comprises at least 10 consecutive nucleotides (e.g., contains 10, 11, 12, 13, 14, 15, 16, 17, 18, 19, 20, or more consecutive nucleotides) of a first adapter (e.g., an Illumina P5 or P7 adapter) of the adapter ligated nucleic acid fragments, or complementary to the first adapter. In some embodiments, the aqueous reaction mixture further comprises a second blocking oligonucleotide that comprises at least 10 consecutive nucleotides (e.g., contains 10, 11, 12, 13, 14, 15, 16, 17, 18, 19, 20, or more consecutive nucleotides) of a second adapter (e.g., an Illumina P5 or P7 adapter) of the adapter ligated nucleic acid fragments, or complementary to the second adapter. In some embodiments, the aqueous reaction mixture further comprises blocking nucleic acids that hybridize to repetitive sequences in at least a portion of the non-target nucleic acid molecules.

In some embodiments, the library of adaptor ligated nucleic acid fragments is a library of adaptor ligated genomic DNA fragments, and the blocking nucleic acid is Cₒt1-DNA, Cₒt2-DNA, or Cₒt3-DNA, or a mixture of two or more of the foregoing. In some embodiments, the bait oligonucleotides comprise RNA oligonucleotides. In some embodiments, the bait oligonucleotides comprise biotin. In some embodiments, the aqueous reaction mixture is in a container, wherein the container further contains: (a) a first immiscible liquid, wherein the first immiscible liquid is less dense than the aqueous reaction mixture, and/or (b) a second immiscible liquid, wherein the second immiscible liquid is more dense than the aqueous reaction mixture. In some cases, the container contains both a first and a second immiscible liquid.

Often the hybridization temperature is in the range of 50° C. to 75° C. In some embodiments the hybridization temperature is about 65° C. In some embodiments the hybridization temperature is about 71° C. In some cases, where labeled bait RNA oligonucleotides are used, the hybridization temperature is higher than typically employed with labeled bait DNA oligonucleotides due to the higher annealing temperature of RNA:DNA hybrids as compared to the annealing of DNA:DNA hybrids having the same sequence. Thus, for example, in some cases labeled bait RNA oligonucleotides may be used with a hybridization temperature of from about 65° C. to about 75° C., whereas labeled bait DNA oligonucleotides may be used with a hybridization temperature of from about 60° C. to about 70° C.

The present method can result in an on-target rate of at least about 65% (e.g., from about 65% to about 85%). In one embodiment carrying out the method using a 10-minute incubation time (e.g., at or at about 65° C.) results in an on-target rate of at least about 65% (e.g., from about 65% to about 70%). In one embodiment carrying out the method using a 30-minute incubation time (e.g., at or at about 65° C.) results in an on-target rate of at least about 75% (e.g., from about 75% to about 80%). In one embodiment carrying out the method using a 120 or 240-minute incubation time (e.g., at or at about 65° C.) results in an on-target rate of at least about 80% (e.g., from about 80% to about 85%).

In some embodiments, the method provides an enrichment of target nucleic acid molecules in the enriched polynucleotide mixture of at least 500-fold, or at least 1,000-fold relative to a sample that is not enriched. In some embodiments, the method provides an enrichment of baited region in the enriched polynucleotide mixture of at least 500-fold, or at least 1,000-fold relative to a sample that is not enriched. In some embodiments, target nucleic acid molecules of the enriched polynucleotide mixture comprise at least about 75% of total target and non-target nucleic acid molecules in the enriched polynucleotide mixture.

In some embodiments, forming the aqueous reaction mixture comprises: i) forming a reaction pre-mixture comprising the nucleic acid sample, water, and bait oligonucleotides; ii) forming a concentrated pre-mixture by reducing the volume of the reaction pre-mixture to a reduced volume, thereby increasing the concentration of target nucleic acid molecules, non-target nucleic acid molecules, and bait oligonucleotides, wherein the reduced volume is less than the volume of the reaction mixture; and iii) contacting the concentrated pre-mixture with a volume of hybridization buffer, wherein the combined volumes of the hybridization buffer and the volume of the concentrated pre-mixture, if any, equal the volume of the aqueous reaction mixture, thereby forming a re-suspended pre-mixture having a volume equal to the volume of the aqueous reaction mixture; and iv) denaturing the target and non-target nucleic acid molecules of the re-suspended pre-mixture by: a) heating the re-suspended pre-mixture to a denaturing temperature; and then b) cooling the re-suspended pre-mixture to a hybridization temperature, thereby forming the aqueous reaction mixture.

In some embodiments, reducing the volume of the reaction pre-mixture to a reduced volume comprises concentrating the pre-mixture to dryness. In some embodiments, the denaturing temperature is at least about 90° C.-99° C., and the denaturing comprises incubating the nucleic acid sample at the denaturing temperature for at least about 5 minutes. In some embodiments, the separating comprises removing aqueous components of the reaction mixture from the immobilized target nucleic acid molecule-bait oligonucleotide complexes, thereby removing nucleic acids and blocking oligonucleotides that are not hybridized to the bait oligonucleotides, and then applying an aqueous wash buffer to the immobilized target nucleic acid molecule-bait oligonucleotide complexes.

In some embodiments, the incubating the aqueous reaction mixture at the hybridization temperature comprises incubating the reaction mixture for at least about 30 minutes at about 65° C. to thereby hybridize at least a portion of the bait oligonucleotides to at least a portion of the target nucleic acids. In some embodiments, the incubating the aqueous reaction mixture at the hybridization temperature comprises incubating the reaction mixture for at least about 45 minutes at about 65° C. to thereby hybridize at least a portion of the bait oligonucleotides to at least a portion of the target nucleic acids. In some embodiments, the incubating the aqueous reaction mixture at the hybridization temperature comprises incubating the reaction mixture for at least about 60 minutes at about 65° C. to thereby hybridize at least a portion of the bait oligonucleotides to at least a portion of the target nucleic acids.

In some embodiments, the incubating the aqueous reaction mixture at the hybridization temperature comprises incubating the reaction mixture for at least about 90 minutes at about 65° C. to thereby hybridize at least a portion of the bait oligonucleotides to at least a portion of the target nucleic acids. In some embodiments, the incubating the aqueous reaction mixture at the hybridization temperature comprises incubating the reaction mixture for at least about 30 minutes and less than about 240 minutes at about 65° C. to thereby hybridize at least a portion of the bait oligonucleotides to at least a portion of the target nucleic acids. In some embodiments, the incubating the aqueous reaction mixture at the hybridization temperature comprises incubating the reaction mixture for between about 10 minutes and 30 about minutes (e.g., about 10 minutes, about 30 minutes, or from 10 minutes to 30 minutes) at about 65° C. to thereby hybridize at least a portion of the bait oligonucleotides to at least a portion of the target nucleic acids.

In some embodiments, an on-target rate of at least about 75% is achieved within a 30 minute incubation of the aqueous reaction mixture at the hybridization temperature. In some embodiments, an on-target rate of at least about 80% is achieved within a 45 minute, 60 minute, 80 minute, 90 minute or 60-90 minute incubation of the aqueous reaction mixture at the hybridization temperature.

In some embodiments, the step of immobilizing on one or more solid surfaced comprises contacting the bait oligonucleotides with beads comprising an affinity agent that specifically binds the label of the bait oligonucleotides. In some embodiments, the immobilizing comprises contacting the bait oligonucleotides with beads comprising capture agent at a temperature of from about 37° C. to about 75° C. for at least about 10 minutes. In some embodiments, the immobilizing comprises contacting the bait oligonucleotides with beads comprising capture agent at a temperature of from about 60° C. to about 70° C. for at least about 20 minutes. In some embodiments, the label of the bait oligonucleotides comprises biotin and the capture agent comprises avidin or streptavidin. In some embodiments, recovering comprises amplifying the immobilized enriched target nucleic acid molecules to produce amplification products thereof, and collecting the amplification products.

### BRIEF DESCRIPTION OF THE DRAWINGS

FIG. 1: Efficiency of hybrid capture versus hybridization time as measured by the methods described in Example 1. Briefly, XGEN® Exome Research Panel v1.0 bait oligonucleotides (Integrated DNA Technologies, Inc.) were hybridized to genomic fragment samples. Quadruplicate samples were hybridized in a concentrated reaction mixture for 10 minutes (samples 1-4), 20 minutes (samples 5-8), 30 minutes (samples 9-12), and 240 minutes (samples 13-16). After hybridization, all samples were treated equivalently. Samples were washed and the target nucleic acids recovered by PCR amplification and sequenced. Four samples were included at each time point for a total of 16 samples. Sample 8 was removed from the analysis due to a failure in the sequencing step. Relative coverage of target nucleic acid reads was plotted as a function of probe GC content for each sample.

FIG. 2: illustrates efficiency of hybrid capture versus hybridization time as measured by the methods described in Example 1. In this Figure, efficiency is determined using the CollectHSMetrics tool in the Picard package. The efficiency is the calculated value of the PCT_USABLE_BASES_ON_TARGET output field using the input variables described below.

FIGS. 3A-D: illustrates a comparison between hybrid capture performance using a manufacturer's protocol as analyzed by high-throughput sequencing (IDT Stock Data from Coriel Sample, NA12878, provided by Integrated DNA Technologies, Inc.) and a fast hybrid capture reagent protocol as described in Example 3. Two different versions of the fast hybrid capture protocol (V1.1 and V1.2) were performed in, each in duplicate (replicates A and B) as described in Example 3.

### DETAILED DESCRIPTION OF THE INVENTION

### I. Overview

The inventors have surprisingly found that the hybridization time of hybrid capture target enrichment methods can be dramatically reduced using a high concentration of the nucleic acid sample, bait oligonucleotides, or both, in the reaction mixture. Improved methods, compositions, instrumentation, and systems based in part on this surprising finding can be used in a wide variety of applications that benefit from target enrichment of nucleic acid samples, including but not limited to, high-throughput sequencing. The methods, compositions, and kits described herein can provide a high on-target rate for enrichment of a sample with a reduced hybridization time as compared to currently available commercial hybrid capture reagents and kits. A high on-target rate can, e.g., provide increased sensitivity and/or specificity for variant (e.g., SNP, CNV, SSLPs, structural variants, etc.) detection, greater coverage, or a combination thereof, for a given depth of sequencing (e.g., 20×).

### II. Definitions

As used herein, the term “aqueous reaction mixture” refers to a solution containing water and one or more components of a reaction mixture. Exemplary components include, but are not limited to, buffering agents, salts, proteins, and nucleic acids. For purposes of this disclosure, the density of an aqueous reaction mixture is the same as the density of water (1 g/cm³ at 4° C.). As used herein, an aqueous reaction mixture is an acellular mixture of heterologous components. The acellular mixture of heterologous components can contain nucleic acid (e.g., genomic nucleic acid fragments) and other components of cellular lysate.

As used herein, the terms “concentration of target and non-target nucleic acid molecules,” “concentration of target nucleic acid molecules and non-target nucleic acid molecules,” and the like, in reference to an aqueous reaction mixture for enrichment of target nucleic acid molecules from a sample containing a plurality of target nucleic acid molecules and a plurality of non-target nucleic acid molecules, refers to the concentration of nucleic acid molecules from a nucleic acid sample. The term is exclusive of the concentration or amount of bait oligonucleotides, blocking oligonucleotides, or blocking nucleic acid (e.g., Cₒt1-DNA), in the aqueous reaction mixture. The target and non-target nucleic acid molecules can be adaptor ligated fragments, e.g., DNA fragments ligated to Illumina, Roche 454 Life Sciences, or Life Technologies adaptors. Alternatively, the target and non-target nucleic acid molecules can be unmodified fragments (e.g., genomic DNA, total RNA, mRNA, cDNA, etc.). In some cases, the target and non-target nucleic acid molecules are in a complex mixture, such as non-isolated nucleic acid molecules in stabilized saliva, whole blood, or a fraction thereof.

As used herein, the term “concentration of bait oligonucleotides in the aqueous reaction mixture” refers to a total concentration of bait oligonucleotides. For example, for an aqueous reaction mixture containing a total amount of 500 ng of 100,000 structurally distinct 200-mer single-stranded DNA bait oligonucleotides in a volume of 2 the concentration is 250 ng/μL or approximately 3.9 pmol/μL. Similarly, the for an aqueous reaction mixture containing 500 ng of a single 200-mer single-stranded DNA bait oligonucleotide in a volume of 2 the concentration is 250 ng/μL or approximately 3.9 pmol/μL.

As used herein, the term “structurally distinct bait oligonucleotides” refers to bait oligonucleotides that are structurally distinct in that they have different nucleotide sequences.

As used herein, the term “enrichment” or “enriched” in reference to target nucleic acid molecules refers to increasing the amount of target nucleic acid molecules relative to the amount of non-target nucleic acid molecules of a sample containing both target and non-target nucleic acid molecules. Generally, enrichment involves removing at least a portion of non-target nucleic acid molecules. Similarly, the term “enrichment” or “enriched” in reference to enrichment of a baited region above genomic background or relative to a sample that is not enriched refers to increasing the proportion of a baited region of a sample of target and non-target nucleic acids above the proportion occurring in the genome or in a genomic sample that has not been subject to a target enrichment method (e.g., hybrid capture, primer extension target enrichment, a molecular inversion probe-based method, or multiplex target-specific PCR).

Enrichment can be measured by a variety of methods known in the art. In an exemplary embodiment, enrichment can be assessed by subjecting a nucleic acid sample to high-throughput sequencing and counting the number of reads of target and non-target nucleic acid sequences. In some cases, the counts are normalized by removing duplicates and/or correcting for amplification bias. Such normalization can be performed by detecting universal molecule identifiers (e.g., molecular barcodes) as, e.g., described in Fu et al. Proc Natl Acad Sci USA. 2011 May 31; 108(22):9026-31. As used herein, unless otherwise indicated, enrichment values refer to the enrichment of a total population of target or baited nucleic acid molecules in a mixture, rather than any one or more individual molecules. For example, in a hypothetical sample containing 1,000 target nucleic acid molecules and 1×10⁷ non-target nucleic acid molecules that is enriched to form an enriched polynucleotide mixture containing 1,000 target nucleic acid molecules and 1,000 non-target nucleic acid molecules, the fold enrichment equals 5,000.5.

The fold by which the baited region of a sample of target and non-target nucleic acids has been enriched above genomic background can be determined using the CollectHSMetrics tool in the Picard package version 2.5.0 (available at, broadinstitute.github.io/picard/command-line-overview.html) to calculate the “FOLD_ENRICHMENT” output parameter using the following input parameters: MINIMUM_MAPPING_QUALITY: 20; MINIMUM_BASE_QUALITY: 20; CLIP_OVERLAPPING_READS: true; METRIC_ACCUMULATION_LEVEL: [ALL_READS]; NEAR_DISTANCE: 100; COVERAGE_CAP: 200; SAMPLE_SIZE: 1000. The CollectHsMetrics tool requires an aligned SAM or BAM file, and bait and target interval files. The bait and interval files designate the bait oligonucleotides and their target nucleic acids used in the hybrid capture reaction. For commercial bait oligonucleotide panels, e.g., from IDT or AGILENT, the bait and interval files can be obtained from the manufacturer. The aligned SAM or BAM files are aligned to a reference sequence. For all enrichment experiments involving capture of human genomic nucleic acid, the reference sequence is the human genome assembly found in GenBank Accession No.: GCA_000001405.23 (GRCh38.p8).

As used herein, the term “target nucleic acid molecule” refers to a nucleic acid molecule (e.g., genomic fragment, cDNA, RNA, mRNA, or a portion thereof) for which enrichment is desired. For example, the target nucleic acid molecules can be molecules that are intended to be a target of a subsequent detection or analysis method, such as high-throughput sequencing. Exemplary target nucleic acid molecules include, but are not limited, to nucleic acid molecules having exact complementarity to a contiguous region of a bait molecule having a length of from about 60 to about 200 contiguous bases, from about 50 to about 150 contiguous bases, or from about 75 to about 300 contiguous bases. Exemplary target nucleic acid molecules can additionally or alternatively include, but are not limited to, nucleic acid molecules that are not exactly complementary to one of the foregoing numbers of contiguous bases of a bait molecule, yet can hybridize to one or more bait oligonucleotides under stringent hybridization conditions (e.g., highly stringent hybridization conditions). For example, the target nucleic acid molecules can have about 70%, 75%, 80%, 85%, 90%, 95%, or 99% exact complementarity to a contiguous region of a bait molecule having a length of from about 15 to about 300 contiguous bases, from about 20 to about 250 contiguous bases, from about 25 to about 230 contiguous bases, from about 30 to about 200 contiguous bases, from about 40 to about 200 contiguous bases, from about 50 to about 200 contiguous bases, from about 60 to about 200 contiguous bases, from about 50 to about 150 contiguous bases, or from about 75 to about 300 contiguous bases. In some cases, capture of target nucleic acid fragments that do not have exact 100% complementarity to a bait molecule can be useful for detecting mutations in a target nucleic acid.

As used herein, “on-target rate” has its normal meaning in the art, and is a measure of hybrid capture performance. Specifically, “on-target rate” refers to a measure of hybrid capture performance based on a proportion of unique high-throughput and on-target sequencing reads generated by high-throughput sequencing after enrichment of target nucleic acids from a background of non-target nucleic acids. Thus, the “on-target rate” is the proportion of aligned, de-duplicated, on-target bases out of the bases available. The bases available are the set of bases that pass the sequencing vendor's quality control filter. “On-target rate” can be calculated from high-throughput sequencing data with the CollectHsMetrics tool in the Picard package version 2.5.0 (available at, broadinstitute.github.io/picard/command-line-overview.html) using the following input parameters: MINIMUM_MAPPING_QUALITY: 20; MINIMUM_BASE_QUALITY:20; CLIP_OVERLAPPING_READS: true; METRIC_ACCUMULATION_LEVEL: [ALL_READS]; NEAR_DISTANCE: 100; COVERAGE_CAP: 200; SAMPLE_SIZE: 1000. The CollectHsMetrics tool requires an aligned SAM or BAM file, and bait and target interval files. The bait and interval files designate the bait oligonucleotides and their target nucleic acids used in the hybrid capture reaction. For commercial bait oligonucleotide panels, e.g., from IDT or AGILENT, the bait and interval files can be obtained from the manufacturer. The aligned SAM or BAM files are aligned to a reference sequence. For all enrichment experiments involving capture of human genomic nucleic acid, the reference sequence is the human genome assembly found in GenBank Accession No.: GCA_000001405.23 (GRCh38.p8). The “on-target rate” is provided in the “PCT_USABLE_BASES_ON_TARGET” field in the output of the CollectHsMetrics tool.

As used herein, “highly stringent hybridization conditions” refers to conditions under which a nucleic acid will hybridize to its target sequence, typically in a complex mixture of nucleic acids, but to no other sequences. Highly stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, highly stringent conditions are selected to be about 5-10° C. lower than the thermal melting point (Tm) for the specific sequence at a defined ionic strength and pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Highly stringent conditions may also be achieved with the addition of destabilizing agents such as formamide. For selective or specific hybridization, a positive signal is at least two times background, preferably 10 times background hybridization.

As used herein, the term “complementary” refers to refers to Watson-Crick or Hoogsteen base pairing between nucleotides units of a nucleic acid molecule. Percent complementarity refers to the percentage of bases of a first nucleic acid molecule (e.g., target nucleic acid molecule) that can form Watson-Crick or Hoogsteen base pairs with a second nucleic acid molecule (e.g., bait oligonucleotide). A nucleic acid molecule (e.g., target nucleic acid molecule) having exact complementarity to a second nucleic acid molecule (e.g., bait oligonucleotide) has 100% complementarity to the second nucleic acid molecule over the specified region of contiguous bases.

As used herein, the term “non-target nucleic acid molecules” refers to nucleic acid molecules for which enrichment is not desired. For example, the non-target nucleic acid molecules can be molecules that are not intended to be a target of a subsequent detection or analysis method, such as high-throughput sequencing. Exemplary non-target nucleic acid molecules include, but are not limited to, genomic fragments containing or consisting of non-protein coding regions of a genome, repetitive genomic DNA, and the like.

As used herein, the term “bait oligonucleotides” refers to oligonucleotides designed to hybridize to target nucleic acids and containing an affinity tag label. Bait oligonucleotides can be DNA, RNA, or DNA/RNA chimeras. Typically, bait oligonucleotides are RNA. The bait oligonucleotides are labeled with an affinity tag label that permits subsequent isolation by a capture agent. An exemplary label is a biotin group (or groups). After hybridization is complete to form the DNA template:bait hybrids, capture is performed with a component having affinity for the bait. For example, streptavidin-magnetic beads can be used to bind the biotin moiety of biotinylated-baits that are hybridized to the desired DNA targets from the nucleic acid sample.

As used herein, the term “affinity tag” refers to first and second members of a specific binding pair (SBP) or ligand-anti-ligand binding pair, where the members of the pair specifically bind to each other. For convenience, the term “affinity tag” is used to refer to the SBP member that is part of the bait oligonucleotide structure, and the term “capture agent” is used to refer to the SBP member that specifically binds the affinity tag. The binding between the members of the binding pair is generally noncovalent, although a covalent (e.g., disulfide) linkage between binding pair members can also be used. In some cases, where a covalent linkage between binding pair members is used, the covalent linkage is reversible. For example, a covalent disulfide linkage can be cleaved with reducing agent.

Binding between specific binding pairs results in the formation of a binding complex, sometimes referred to as a ligand/antiligand complex or simply as ligand/antiligand. Exemplary binding pairs include, but are not limited to: (a) a nucleic acid aptamer and protein; (b) biotin-avidin, biotin-streptavidin, biotin-Neutravidin, biotin-Tamavidin, streptavidin binding peptide-streptavidin, or glutathione-glutathione S-transferase binding pairs and the like; (c) hormone-hormone binding protein; (d) receptor-receptor agonist or antagonist; (e) lectin-carbohydrate; (f) thio (—S—) or thiol (—SH) containing binding member pairs capable of forming an intramolecular disulfide bond; and (g) complementary metal chelating groups and a metal (e.g., metal chelated by the binding pairs nitrilotriacetate (NTA) and a 6×-His tag). Specific binding pair members need not be limited to pairs of single molecules. For example, a single ligand can be bound by the coordinated action of two or more antiligands.

In the context of the binding of an affinity tag label of a bait oligonucleotide to the capture agent (e.g., of a functionalized solid support), the terms “specific binding,” “specifically binds,” and the like refer to the preferential association of capture agent with a bait oligonucleotide bearing a particular target affinity tag label in comparison to a bait oligonucleotide lacking the affinity tag. Specific binding between a capture agent and affinity tag generally means an affinity of at least 10⁻⁶ M⁻¹ (i.e., an affinity having a lower numerical value than 10⁻⁶ M⁻¹ as measured by the dissociation constant K_{d}). Affinities greater than 10⁻⁸ M⁻¹ are preferred. Specific binding can be determined using any assay binding known in the art.

As used herein, the term “blocking oligonucleotide” refers to an oligonucleotide that hybridizes to a nucleic acid molecule having a sequence that is present, or suspected of being present, in a nucleic acid sample. Exemplary blocking oligonucleotides hybridize to high-throughput library adaptor sequences present in all adaptor ligated fragments of a nucleic acid sample. Further exemplary blocking oligonucleotides are described in WO 2014/008,447, the contents of which are hereby incorporated by reference in the entirety for all purposes.

As used herein, the term “blocking nucleic acid” refers to a mixture of nucleic acid (e.g., DNA) fragments that are neither adaptor ligated, nor labeled with the label that that permits subsequent capture of bait oligonucleotides, and are enriched in non-target nucleic acid molecules, or a complement thereof. Blocking nucleic acid can be incorporated into a hybrid capture method, e.g., where the nucleic acid sample is a library of adaptor ligated fragments. Such blocking nucleic acid can hybridize to repetitive sequences in the library to reduce non-specific hybridization of bait oligonucleotides to, and thus reduce capture of, the repetitive sequences. Moreover, blocking nucleic acid can reduce capture of repetitive sequences by hybridization to a nucleic acid fragment that contains both a target nucleic acid region and a repetitive region.

In exemplary embodiments, the nucleic acid sample is a library of adaptor ligated genomic fragments having common repetitive DNA fragments, such as LINE elements, SINE elements, Alu repeats, etc. In such cases, the blocking nucleic acid can be Cₒt−1 DNA, Cₒt−2 DNA, Cₒt−3 DNA, sheared salmon sperm DNA, or a mixture of two, three, or four of the foregoing, or a composition of blocking nucleic acid described in U.S. Pat. No. 7,833,713, the contents of which are hereby incorporated by reference in the entirety for all purposes. In an exemplary embodiment, the blocking nucleic acid is Cₒt−1 DNA.

As used herein, the terms “Cₒt−1 DNA,” “Cₒt−2 DNA,” and “Cₒt−2 DNA,” refer to mixtures of genomic DNA of a single species (e.g., human) that has been denatured and then renatured at an initial DNA concentration (Cₒ) and for a period of time (t), where the product of Cₒ and t=1, 2, or 3 respectively. Cₒt DNA having a low value (e.g., from 1 to 3) is enriched in repetitive genomic DNA fragments, such as LINE elements, SINE elements, Alu repeats, etc. In an exemplary embodiment, the Cₒt−1 DNA is human placental Cₒt−1 DNA, where at least 50% of the fragments are between 50 and 300 bp in length (e.g., at least 50% of the fragments are from 50 to 300 bp in length). In another exemplary embodiment, the Cₒt−1 DNA is human placental Cₒt−1 DNA, where the DNA is enriched in repetitive sequences of 50 to 100 bp in length. In another exemplary embodiment, the Cₒt−1 DNA is human placental Cₒt−1 DNA, where at least 50% of the fragments are between 50 and 300 bp in length and the DNA is enriched in repetitive sequences of 50 to 100 bp in length.

Proprietary mixtures of Cₒt DNA are available, such as COT-1 DNA®. Commercial procedures for Cₒt−1 DNA preparation iterate denaturation and re-annealing of genomic DNA, and are monitored by enrichment for Alu elements (three-fold excess over the corresponding level in the normal genome) and L1 elements (four-fold excess over the corresponding level in the normal genome). Current quality control procedures do not determine the precise composition or sequence of Cot-1 DNA.

As used herein, the term “immiscible liquid” refers to a liquid having a solubility in water of less than 100 parts per billion (ppb). In some cases, immiscible liquid also refers to a liquid having a solubility in a second mutually immiscible liquid of less than about 10% (w/w, w/v, or v/v), or less than 1% (w/w, w/v, or v/v). The relative immiscibility of a pair of liquid solvents, or of each component of a three-phase system, can be empirically determined, or can be estimated using various solubility parameters. For example, the Hildebrand solubility parameter can be used to estimate the relative immiscibility of liquids, where a large difference (e.g., at least 5, 10, 15, or 20 MPa) between liquids can indicate mutual immiscibility. See, e.g., Adams D., Dyson; P., Tavener, S. Chemistry in Alternative Reaction Media 2004, John Wiley & Sons, incorporated herein by reference. Various immiscible liquids, and compositions and articles of manufacture containing such immiscible liquids, as well as methods of their use are described in the co-ending U.S. provisional application entitled “Methods and Compositions for Low Volume Liquid Handling,” U.S. Application No. 62/263,543 (filed on Dec. 4, 2015), the contents of which are incorporated by reference in the entirety.

As used herein, the term “more dense” in the context of a density of an immiscible liquid in comparison to an aqueous reaction mixture refers to an immiscible liquid that is at least 25% more dense than water in terms of g/cm³ at the same temperature and pressure.

As used herein, the term “less dense” in the context of a density of an immiscible liquid in comparison to an aqueous reaction mixture refers to an immiscible liquid that is less than about 99% of the density of water in terms of g/cm³ at the same temperature and pressure.

As used herein, the term “dryness” in the context of the claims refers to a degree of concentration, such that a concentrated reaction pre-mixture contains less than about 1% water by weight.

### III. Compositions

Described herein are aqueous reaction mixtures for hybrid capture enrichment of nucleic acid samples. In certain aspects, these aqueous reaction mixtures can provide a high level of target enrichment with a dramatically reduced hybridization time.

In one aspect, the aqueous reaction mixture is a mixture for enrichment of target nucleic acid molecules from a nucleic acid sample containing a plurality of target and non-target nucleic acid molecules. In one embodiment, the mixture contains: a) a plurality of structurally distinct bait oligonucleotides, wherein the structurally distinct bait oligonucleotides are complementary to the plurality of target nucleic acid molecules in the sample; b) the nucleic acid sample containing the plurality of target nucleic acid molecules and the plurality of non-target nucleic acid molecules; and water.

The volume of the aqueous reaction mixture can be less than about 10 μL, less than about 7 μL, less than about 5 μL, less than about 4 μL, less than about 3 μL, or about 2 μL. In some cases, the volume of the aqueous reaction mixture is no less than about 0.1 μL and no more than about 10 μL. In some cases, the volume of the aqueous reaction mixture is no less than about 0.5 μL and no more than about 10 μL. In some cases, the volume of the aqueous reaction mixture is no less than about 0.5 μL and no more than about 5 μL. In some cases, the volume of the aqueous reaction mixture is no less than about 0.5 μL and no more than about 3 μL. In some cases, the volume of the aqueous reaction mixture is no less than about 1 μL and no more than about 5 μL. In some cases, the volume of the aqueous reaction mixture is no less than about 1 μL and no more than about 3 μL. In some cases, the volume of the aqueous reaction mixture is no less than about 1.5 μL and no more than about 5 μL. In some cases, the volume of the aqueous reaction mixture is no less than about 1.5 μL and no more than about 3 μL. In some cases, the volume of the aqueous reaction mixture is from about 0.1 μL to about 5 μL, from about 0.5 μL to about 5 μL, from about 0.75 μL to about 4 μL, or from about 1 μL to about 3 μL.

The sample containing a plurality of target nucleic acid molecules and a plurality of non-target nucleic acid molecules can be a sample of nucleic acid from any suitable source. In some embodiments, the nucleic acid sample is a sample of nucleic acid from a single organism (e.g., a single mammal, rodent, non-human primate, or human). In some embodiments, the nucleic acid sample is a sample of nucleic acid from a single tissue or organ. For example, the sample can be from blood (e.g., whole blood or a fraction thereof), plasma, or tissue from a biopsy. The nucleic acid can be unpurified or partially purified. The nucleic acid can be isolated, purified, reverse transcribed, polymerized, amplified, digested, or ligated prior to introduction into the aqueous reaction mixture. The sample can be a sample of DNA, genomic DNA, total RNA, rDNA, mtDNA, cDNA, RNA, mRNA, miRNA, rRNA and the like.

As described herein, target nucleic acid molecules can be any nucleic acid molecule that is desired to be enriched by the hybrid capture reaction. For instance, in a whole exome sequencing method (a method for sequencing all the protein-coding genes in a genome), a target nucleic acid molecule can contain an exon, or fragment thereof. As another example, in a high-throughput sequencing method directed to sequencing cancer markers, a target nucleic acid molecule can contain a sequence diagnostic of cancer risk, disease progression or remission, tumor state, and/or prognosis.

In an exemplary embodiment, the target nucleic acid molecules include nucleic acid molecules that contain a genomic fragment corresponding to at least a portion (e.g., a portion of sufficient length and complementarity to be captured by a bait oligonucleotide under hybrid capture conditions) of a hereditary cancer risk gene. Such hereditary cancer risk genes, can include, but are not limited those genes selected from the group consisting of ATM, BARD1, BRCA1, BRCA2, BRIP1, CDH1, CHEK2, EPCAM, MLH1, MSH2, MSH6, NBN, PALB2, PMS2, PTEN, RAD51C, RAD51D, STK11, and TP53. In another exemplary embodiment, the target nucleic acid molecules consist of nucleic acid molecules that contain a genomic fragment corresponding to at least a portion (e.g., a portion of sufficient length and complementarity to be captured by a bait oligonucleotide under hybrid capture conditions) of one of the following genes ATM, BARD1, BRCA1, BRCA2, BRIP1, CDH1, CHEK2, EPCAM, MLH1, MSH2, MSH6, NBN, PALB2, PMS2, PTEN, RAD51C, RAD51D, STK11, and TP53.

In another exemplary embodiment, the target nucleic acid molecules include nucleic acid molecules that contain a genomic fragment corresponding to at least a portion (e.g., a portion of sufficient length to be captured by a bait oligonucleotide under hybrid capture conditions) of a gene selected from the group consisting of the gene targets of one or more of the following commercial bait oligonucleotide mixtures: XGEN® Exome Research Panel v1.0, XGEN® Acute Myeloid Leukemia (AML) Cancer Panel v1.0, XGEN® Pan-Cancer Panel v1.0, XGEN® Pan-Cancer Panel v1.5, XGEN® Inherited Diseases Panel v1.0, Sure SelectXT Clinical Research Exome, and Sure SelectXT2 Clinical Research Exome. Further exemplary bait oligonucleotides are described in U.S. 2010/0029498, the contents of which are hereby incorporated by reference in the entirety for all purposes.

The XGEN® Exome Research Panel v1.0 consists of 429,826 different DNA oligonucleotide probes spanning 39 Mb of target regions of the human genome, targeting 19,396 genes of the human genome, and covering 51 Mb of end-to-end tiled space. The bait oligonucleotides in the XGEN® Exome Research Panel v1.0 can be accessed on the world wide web at web.archive.org/web/20160325181826/http://www.idtdna.com/pages/docs/default-source/xgen-libraries/xGen-Lockdown-Panels/xgen-exome-research-panel-probes.bed?sfvrsn=4. The genes targeted by the XGEN® Exome Research Panel v1.0 can be accessed on the world wide web at web.archive.org/web/20160325181826/http://www.idtdna.com/pages/docs/default-source/xgen-libraries/xGen-Lockdown-Panels/xgen-exome-research-panel-gene-list.txt?sfvrsn=4.

The XGEN® Acute Myeloid Leukemia Cancer Panel v1.0 consists of 11,743 xGen Lockdown DNA oligonucleotide probes, spanning 1.19 Mb of the human genome, for targeted enrichment of approximately 260 genes associated with the AML. The bait oligonucleotides in the XGEN® Acute Myeloid Leukemia Cancer Panel v1.0 can be accessed on the world wide web at web.archive.org/web/20160326003009/http://www.idtdna.com/pages/docs/default-source/xgen-libraries/xGen-Lockdown-Panels/xgen-aml-probes.bed?sfvrsn=2. The genes targeted by the XGEN® Acute Myeloid Leukemia Cancer Panel v1.0 can be accessed on the world wide web at web.archive.org/web/20160326003009/http://www.idtdna.com/pages/docs/default-source/xgen-libraries/xGen-Lockdown-Panels/xgen-aml-cancer-panel---gene-list.xlsx?sfvrsn=2.

The XGEN® Inherited Diseases Panel v1.0 consists of 116,355 xGen Lockdown DNA oligonucleotide probes, spanning 11.1 Mb of the human genome, designed for targeted enrichment of 4503 genes and 181 SNPs associated with inherited diseases. The bait oligonucleotides in the XGEN® Inherited Diseases Panel v1.0 can be accessed on the world wide web at web.archive.org/web/20160326003015/http://www.idtdna.com/pages/docs/default-source/xgen-libraries/xGen-Lockdown-Panels/xgen-inherited-diseases-probes.bed?sfvrsn=4. The genes targeted by the XGEN® Inherited Diseases Panel v1.0 can be accessed on the world wide web at web.archive.org/web/20160326003015/http://www.idtdna.com/pages/docs/default-source/xgen-libraries/xGen-Lockdown-Panels/xgen-inherited-diseases-gene-list.xlsx?sfvrsn=4.

The XGEN® Pan-Cancer Panel v1.5 consists of 7816 xGen Lockdown® DNA oligonucleotide probes, spanning 800 kb of the human genome, that capture 127 significantly mutated genes implicated across 12 tumor tissue types. The 127 genes targeted in the XGEN® Pan-Cancer Panel v1.5 are listed in the following Table, which can be accessed on the world wide web at web.archive.org/web/20160603171626/https://www.idtdna.com/pages/docs/default-source/xgen-libraries/xGen-Lockdown-Panels/xgen-pan-cancer-gene-list.xlsx?sfvrsn=4:

| | | | | | | | | | | |
|---|---|---|---|---|---|---|---|---|---|---|
| ACVR1B | ATRX | CDKN1B | ELF3 | FOXA1 | KIT | KMT2D | PCBP1 | RB1 | SOX17 | TSHZ2 |
| ACVR2A | AXIN2 | CDKN2A | EP300 | FOXA2 | KRAS | MTOR | PDGFRA | RPL22 | SOX9 | TSHZ3 |
| AJUBA | B4GALT3 | CDKN2C | EPHA3 | GATA3 | LIFR | NAV3 | PHF6 | RPL5 | SPOP | U2AF1 |
| AKT1 | BAP1 | CEBPA | EPHB6 | H3F3C | LRRK2 | NCOR1 | PIK3CA | RUNX1 | STAG2 | USP9X |
| APC | BRAF | CHEK2 | EPPK1 | HGF | MALAT1 | NF1 | PIK3CG | SETBP1 | STK11 | VEZF1 |
| AR | BRCA1 | CRIPAK | ERBB4 | HIST1H1C | MAP2K4 | NFE2L2 | PIK3R1 | SETD2 | TAF1 | VHL |
| ARHGAP35 | BRCA2 | CTCF | ERCC2 | HIST1H2BD | MAP3K1 | NFE2L3 | POLQ | SF3B1 | TBL1XR1 | WT1 |
| ARID1A | CBFB | CTNNB1 | EZH2 | IDH1 | MAPK8IP1 | NOTCH1 | PPP2R1A | SIN3A | TBX3 | |
| ARID5B | CCND1 | DNMT3A | FBXW7 | IDH2 | MECOM | NPM1 | PRX | SMAD2 | TET2 | |
| ASXL1 | CDH1 | EGFR | FGFR2 | KDM5C | MIR142 | NRAS | PTEN | SMAD4 | TGFBR2 | |
| ATM | CDK12 | EGR3 | FGFR3 | KDM6A | KMT2B | NSD1 | PTPN11 | SMC1A | TLR4 | |
| ATR | CDKN1A | EIF4A2 | FLT3 | KEAP1 | KMT2C | PBRM1 | RAD21 | SMC3 | TP53 | |

The list of bait oligonucleotides in the XGEN® Pan-Cancer Panel v1.5 can be accessed on the world wide web at web.archive.org/web/20160603171626/https://www.idtdna.com/pages/docs/default-source/xgen-libraries/xGen-Lockdown-Panels/xgen-pan-cancer-probes.bed?sfvrsn=4.

Generally, the target nucleic acid molecules contain a region complementary to a bait oligonucleotide, where the complementarity is sufficient to allow sequence specific hybridization, and thus capture, under typical hybrid capture conditions. In a non-limiting aspect, the target nucleic acid molecules are at least 70%, at least 80%, at least 85%, at least 90%, at least 95%, at least 96%, at least 97%, at least 98%, at least 98%, at least 99%, or 100% complementary (e.g., exactly complementary) to a region of contiguous bases of a bait oligonucleotide. In another non-limiting aspect, the target nucleic acid molecules are from about 70% to 100%, from about 75% to about 100%, from about 80% to about 100%, from about 85% to about 100%, from about 90% to about 100%, or from about 95% to 100% complementary (e.g., exactly complementary) to a region of contiguous bases of a bait oligonucleotide. The region of contiguous bases of the bait oligonucleotide can have a length in nucleotides of from about 15 to about 300; from about 20 to about 250; from about 25 to 230; from about 30 to about 200; from about 40 to about 200; from about 50 to about 200; or from about 60 to about 200.

The concentration of target and non-target nucleic acid molecules of the sample in the aqueous reaction mixture is at least about 50 ng/μL; at least about 75 ng/μL; at least about 100 ng/μL; at least about 125 ng/μL; at least about 150 ng/μL; at least about 175 ng/μL; at least about 200 ng/μL; at least about 225 ng/μL; at least about 250 ng/μL; at least about 275 ng/μL; at least about 300 ng/μL; at least about 325 ng/μL; at least about 350 ng/μL; at least about 375 ng/μL; at least about 400 ng/μL; at least about 425 ng/μL; at least about 450 ng/μL; at least about 475 ng/μL; at least about 500 ng/μL; at least about 600 ng/μL; at least about 700 ng/μL; at least about 750 ng/μL; at least about 800 ng/μL; at least about 900 ng/μL; or at least about 1,000 ng/μL. In some cases, the concentration of target and non-target nucleic acid molecules of the sample in the aqueous reaction mixture is about 50 ng/μL; 75 ng/μL; 100 ng/μL; 125 ng/μL; 150 ng/μL; 175 ng/μL; 200 ng/μL; 225 ng/μL; 250 ng/μL; 275 ng/μL; 300 ng/μL; 325 ng/μL; 350 ng/μL; 375 ng/μL; 400 ng/μL; 425 ng/μL; 450 ng/μL; 475 ng/μL; 500 ng/μL; 600 ng/μL; 700 ng/μL; 750 ng/μL; 800 ng/μL; 900 ng/μL; or 1,000 ng/μL.

In some cases, the concentration of target and non-target nucleic acid molecules of the sample in the aqueous reaction mixture is not less than about 50 ng/μL and not more than about 500 ng/μL. In some cases, the concentration of target and non-target nucleic acid molecules of the sample in the aqueous reaction mixture is from about 100 ng/μL to about 300 ng/μL. In some cases, the concentration of target and non-target nucleic acid molecules of the sample in the aqueous reaction mixture is about 250 ng/μL.

In some cases, the concentration of target and non-target nucleic acid molecules of the sample in the aqueous reaction mixture is not less than about 100 ng/μL and not more than about 2,500 ng/μL. In some cases, the concentration of target and non-target nucleic acid molecules of the sample in the aqueous reaction mixture is not less than about 100 ng/μL and not more than about 2,500 ng/μL. In some cases, the concentration of target and non-target nucleic acid molecules of the sample in the aqueous reaction mixture is not less than about 200 ng/μL and not more than about 2,500 ng/μL. In some cases, the concentration of target and non-target nucleic acid molecules of the sample in the aqueous reaction mixture is not less than about 250 ng/μL and not more than about 2,500 ng/μL. In some cases, the concentration of target and non-target nucleic acid molecules of the sample in the aqueous reaction mixture is not less than about 300 ng/μL and not more than about 2,500 ng/μL. In some cases, the concentration of target and non-target nucleic acid molecules of the sample in the aqueous reaction mixture is not less than about 400 ng/μL and not more than about 2,500 ng/μL. In some cases, the concentration of target and non-target nucleic acid molecules of the sample in the aqueous reaction mixture is not less than about 600 ng/μL and not more than about 2,500 ng/μL.

In some cases, the concentration of target and non-target nucleic acid molecules of the sample in the aqueous reaction mixture is not less than about 100 ng/μL and not more than about 1,500 ng/μL. In some cases, the concentration of target and non-target nucleic acid molecules of the sample in the aqueous reaction mixture is not less than about 200 ng/μL and not more than about 1,500 ng/μL. In some cases, the concentration of target and non-target nucleic acid molecules of the sample in the aqueous reaction mixture is not less than about 250 ng/μL and not more than about 1,500 ng/μL. In some cases, the concentration of target and non-target nucleic acid molecules of the sample in the aqueous reaction mixture is not less than about 300 ng/μL and not more than about 1,500 ng/μL. In some cases, the concentration of target and non-target nucleic acid molecules of the sample in the aqueous reaction mixture is not less than about 400 ng/μL and not more than about 1,500 ng/μL. In some cases, the concentration of target and non-target nucleic acid molecules of the sample in the aqueous reaction mixture is not less than about 600 ng/μL and not more than about 1,500 ng/μL.

In some cases, the concentration of target and non-target nucleic acid molecules of the sample in the aqueous reaction mixture is not less than about 100 ng/μL and not more than about 1,200 ng/μL. In some cases, the concentration of target and non-target nucleic acid molecules of the sample in the aqueous reaction mixture is not less than about 200 ng/μL and not more than about 1,200 ng/μL. In some cases, the concentration of target and non-target nucleic acid molecules of the sample in the aqueous reaction mixture is not less than about 250 ng/μL and not more than about 1,200 ng/μL. In some cases, the concentration of target and non-target nucleic acid molecules of the sample in the aqueous reaction mixture is not less than about 300 ng/μL and not more than about 1,200 ng/μL. In some cases, the concentration of target and non-target nucleic acid molecules of the sample in the aqueous reaction mixture is not less than about 400 ng/μL and not more than about 1,200 ng/μL. In some cases, the concentration of target and non-target nucleic acid molecules of the sample in the aqueous reaction mixture is not less than about 600 ng/μL and not more than about 1,200 ng/μL.

In one aspect, the sample is a DNA sample (e.g., genomic DNA). The DNA sample can be fragmented to a specified size range of fragments (e.g., genomic fragments). In some embodiments, the fragments are from 200 to 500 bp in length, however the preferred range of fragments depends on the particular application and/or high-throughput sequencing methodology used for downstream processing. DNA can be fragmented by sonication, nebulization, restriction digestion, DNAse digestion, and the like. Furthermore, fragmented DNA can include internal breaks (e.g., nicks) within one of the two complementary strands that do not result in complete breakage of the double-stranded DNA structure. Such internal breaks can be repaired using a DNA polymerase having nick-translation activity in the presence of dNTPs (e.g., T4 DNA polymerase or Large (Klenow) Fragment of DNA Polymerase I, among others) or in the presence of a suitable ligase in the presence of ATP (e.g., T4 DNA ligase).

DNA fragments can be enzymatically treated to provide blunt end termini, or termini having a pre-determined terminal overhang (e.g., a 3′ A overhang) that is complementary to a pre-determined adaptor overhang (e.g., a 3′ T overhang). After treatment, the fragments can be ligated to adaptors that facilitate high-throughput sequencing. Adaptors can be designed to include different types of termini. This design is chosen to provide a single copy of double-stranded adaptor for each end of the resultant templates.

For fragments enzymatically treated to include flush-ended termini, adaptors are designed to include a first terminus having a flush end and a second terminus having an overhang end. For such adaptors, the second terminus is further designed to include one or more features that preclude ligation to other adaptors (for example, lacking a ligase-competent substrate, such as a 5′-phosphate group, 3′-hydroxyl group, and/or sequence complementarity, among others). For fragments enzymatically treated to include single-nucleotide termini, adaptors can be designed to include a first terminus having a complementary single-nucleotide overhang and a second terminus having a different type of end. Like that described above, the second terminus of the latter adaptors can be preferably designed to include one or more features that precludes ligation to other adaptors.

The oligonucleotide composition of adaptors can include conventional nucleobases, wherein the internucleotidyl linkages are conventional phosphodiester moieties. The adaptors can include chemical groups that display Tm-enhanced properties, as further explained below. The oligonucleotide adaptors can range in length from about 15 nucleotides to about 75 nucleotides.

For certain high-throughput sequencing applications, “barcode” sequences can be appended to the target and non-target nucleic acid molecules to enable multiplex sequencing in massively parallel sequencing experiments. For this purpose, adaptors can include a plurality of nucleotide positions having mixed nucleobase compositions (for example, a mixture of two or more canonical nucleobases at a particular position(s)), including “universal” nucleobase compositions (for example, inosine, 3-nitropyrrole, 5-nitroindole, among others) that represent the barcode sequence tags. As used herein, a “universal nucleobase” refer to a nucleobase that exhibits the ability to replace any of the four normal nucleobases without significantly destabilizing neighboring base-pair interactions. When such mixed nucleobase compositions, including universal nucleobase compositions, are present in adaptors, they occupy a plurality of substantially contiguous nucleotide positions ranging in lengths preferably from about 5 to about 12 nucleotides. Preferably, the plurality of substantially contiguous nucleotide positions that includes these nucleobases is located within the oligonucleotide at a central position away from the termini.

The primary sequence composition of adaptors can depend upon a number of considerations. One consideration is the high-throughput sequencing platform used for the massively parallel sequencing experiments. For example, the commercially available automated instrumentation used for high-throughput sequencing applications have different libraries of templates containing different adaptors, so the selection of primary sequence compositions for any given commercial high-throughput sequencing instrumentation platform will depend upon that criterion. Another consideration is the primary sequence compositional design of blocking oligonucleotides.

Adaptors can be appended to (e.g., ligated to) any type of target and non-target nucleic acid molecules. In some cases, the adaptors are appended to fragments of the DNA, genomic DNA, rDNA, mtDNA, cDNA, RNA, mRNA, miRNA, rRNA and the like, in a manner that is sequence independent. Thus, both target and non-target nucleic acid molecules are adaptor appended with the same or similar frequency. In other cases, adaptors are appended in a sequence dependent manner. Adaptors can be appended in a sequence dependent fashion using one or more primer that contain adaptor sequence, or a portion thereof, at the 5′ end.

The aqueous reaction mixture can contain bait oligonucleotides. The baits are designed to hybridize to the target nucleic acid molecules within the sample of nucleic acids and are usually 60-200 bases in length and further are modified to contain a label that permits subsequent capture of these probes. One common capture method incorporates a biotin group (or groups) on the baits, although any label for which a specifically binding capture agent is available can be used.

The term “specifically binding” refers to a preferential association between a label-bearing nucleic acid molecule and capture agent as compared to a non-label-bearing nucleic acid molecule and the capture agent. It is recognized that a certain degree of non-specific interaction may occur between a capture agent and unlabeled nucleic acid molecules. Nevertheless, specific binding, may be distinguished as mediated through specific recognition of the label by the capture agent. Specific binding results in a much stronger association between the labeled nucleic acid molecules and capture agent than between the capture agent and nucleic acid molecules lacking the label. Specific binding typically results in greater than 10-fold, for example greater than 100-fold, greater than 1,000-fold, greater than 10,000-fold, greater than 100,000-fold, or greater than 1,000,000 fold preferential binding of labeled nucleic acid molecule to capture agent as compared to unlabeled nucleic acid molecules.

After hybridization is complete to form the DNA template:bait hybrids, capture is performed with a component having a specific affinity for the bait. For example, streptavidin-magnetic beads can be used to bind the biotin moiety of biotinylated-baits that are hybridized to the desired nucleic acid targets from the pool of target and non-target nucleic acid molecules. Washing removes unbound nucleic acid molecules, reducing the complexity of the retained material. The retained material, or amplification products thereof, is then collected from the magnetic beads and, e.g., introduced into automated sequencing processes.

The nucleic acid portion of bait oligonucleotides can contain or consist of DNA, RNA, or a combination thereof. In some cases, the bait oligonucleotides contain one or more nucleotide modifications. For example, the bait oligonucleotides can contain a nucleotide modification that increases the melting temperature of a [bait oligonucleotide]:[target nucleic acid molecule] complex. Examples, of such modifications include, but are not limited to locked nucleic acid groups, bicyclic nucleic acid groups, C5-modified pyrimidine groups, peptide nucleic acid groups, and combinations thereof. In a non-limiting aspect, the bait oligonucleotides are capable of hybridizing or binding to target nucleic acid molecules that are at least 70%, at least 80%, at least 85%, at least 90%, at least 95%, at least 96%, at least 97%, at least 98%, at least 98%, at least 99%, or 100% identical to a complementary sequence of a bait oligonucleotide. In an exemplary embodiment, the bait oligonucleotides are 120 bp DNA molecules that are covalently linked to a biotin moiety at the 5′ end.

The concentration of bait oligonucleotides can be at least about 0.2 pmol/μL, at least about 0.3 pmol/μL, at least about 0.4 pmol/μL, at least about 0.5 pmol/μL, at least about 0.6 pmol/μL, at least about 0.7 pmol/μL, at least about 0.8 pmol/μL, at least about 0.9 pmol/μL, at least about 1 pmol/μL, or more. In some cases, the concentration of bait oligonucleotides is not less than about 0.2 pmol/μL and not more than about 5 pmol/μL. In some cases, the concentration of bait oligonucleotides in the aqueous reaction mixture can be at least 0.75 pmol/μL. For example, the concentration of bait oligonucleotides in the aqueous reaction mixture can be from about 0.5 pmol/μL to about 2 pmol/μL, from about 0.6 pmol/μL to about 2 pmol/μL, from about 0.7 pmol/μL to about 2 pmol/μL, from about 0.75 pmol/μL to about 2 pmol/μL, from about 1 pmol/μL to about 2 pmol/μL, or about 1.5 pmol/μL.

The aqueous reaction mixture can contain blocking oligonucleotides. Blocking oligonucleotides are applicable where the target and non-target nucleic acid molecules are adaptor-appended (e.g., ligated) fragments. Because the pool fragments contain identical terminal adaptor sequences on every fragment, the adaptor sequences are present at a very high effective concentration(s) in the aqueous reaction mixture. Consequently, unrelated nucleic acid molecules can anneal to each other through their termini, thereby resulting in a “daisy chain” of otherwise unrelated DNA fragments being linked together. If one of these linked fragments is a target nucleic acid fragment, it therefore contains a sequence complementary to a bait oligonucleotide. The target nucleic acid fragment can hybridize to the bait oligonucleotide, and the entire daisy chain can be captured. In this way, capture of a single target fragment can bring along a large number of non-target fragments, which reduces the overall efficiency of enrichment for the desired fragment.

This class of unwanted capture event can be reduced by adding an excess of single-stranded adaptor sequences to the hybridization reaction as blocking oligonucleotides. In some cases, the blocking oligonucleotides differ from the single stranded adaptor sequences by containing one or more nucleotide modifications. For example, the blocking oligonucleotides can contain a nucleotide modification that increases the melting temperature of a blocking oligonucleotide:adaptor complex. Examples, of such modifications include, but are not limited to locked nucleic acid groups, bicyclic nucleic acid groups, C5-modified pyrimidine groups, peptide nucleic acid groups, and combinations thereof.

In some cases, the adaptor sequences can contain one or more regions that are not fully defined or are otherwise variable, e.g., a degenerate region. Such regions can be useful as barcodes for sample tagging, sourcing, molecular counting, tracking, sorting, de-duplification, removal of amplification bias, error correcting, etc. In some cases, a region of the blocking oligonucleotides that corresponds to a variable region of the adaptor sequences can contain one or more, or all, universal bases capable of base pairing with any “N” (A, C, G, T), or partially universal bases. Such universal, or partially universal, bases include, but are not limited to, inosine, 5-nitroindole, 2-amino purine, nebularine, and the like.

In some cases, the blocking oligonucleotides can be modified at a 3′ end to prevent extension by a polymerase in post-capture sample processing. A wide variety of suitable 3′ end modifications are known in the art, including, but not limited to, an optionally substituted C₁-C₂₄ alkyl diol (e.g., a 3′-hexanediol modification), an optionally substituted C₂-C₂₄ alkenyl diol, an optionally substituted C₂-C₂₄ alkynyl diol, a minor groove binder (MGB), an amine (NH₂), PEG, PO₄, and combinations thereof.

Generally, blocking oligonucleotides are within 1, 2, 3, 4, 5, 6, 7, 8, 9, or 10 nucleotides of the length of the adaptor sequences. In an exemplary embodiment, the blocking oligonucleotides are matched (e.g., exactly matched) in length and sequence to adaptors of an adaptor ligated library of nucleic acid fragments. In another exemplary embodiment, the blocking oligonucleotides contain degenerate bases (e.g., deoxyinosine) at regions corresponding to barcode regions of the adaptors, and are otherwise exactly matched in length and sequence to adaptor sequences. In one exemplary embodiment, the blocking oligonucleotides are matched in length and sequence to the adaptor sequences of an adaptor ligated nucleic acid fragment library, contain deoxyinosine at regions corresponding to barcode regions of the adaptors, and contain a 3′ blocking moiety to prevent extension or amplification by polymerase in downstream processes. Additional blocking oligonucleotides include but are not limited to those described in WO 2014/008,447.

The aqueous reaction mixture can contain blocking nucleic acid. Blocking nucleic acid can be used to reduce unwanted capture of non-target nucleic acid fragments containing repetitive sequence. For example, the repetitive endogenous DNA elements, such as an Alu sequence, SINE sequence, or LINE sequence, present in one DNA fragment in a complex pool can hybridize to another similar element present in another unrelated DNA fragment. These fragments, which may originally derive from very different locations within the genome, become linked during the hybridization process of the enrichment method. If one of these linked fragments is a target nucleic acid fragment, it therefore contains a sequence complementary to a bait oligonucleotide. The target nucleic acid fragment can hybridize to the bait oligonucleotide, and the entire linked chain can be captured. In this way, capture of a single target fragment can bring along a large number of non-target fragments, which reduces the overall efficiency of enrichment for the desired fragment. This class of non-target nucleic acid molecules can be reduced by adding an excess of unlabeled repeat elements to the hybridization reaction. Most commonly, Cₒt DNA (Cₒt−1, Cₒt−2, Cₒt−3, or a mixture thereof) is added to the hybridization reaction, which binds Alu, LINE, and other repeat sites in the target and blocks the ability of nucleic acid fragments to interact with each other on that basis. Generally, the species of the Cₒt DNA is matched to the species of the organism from which the nucleic acid fragments are derived. Thus, human Cₒt DNA is used for nucleic acid samples derived from a human.

Aqueous reaction mixtures described herein can be in contact with one or more immiscible liquids. Such immiscible liquids can provide improved liquid handling, higher thermal inertia, improved reaction temperature and/or composition control, and reduced loss of sample. In some cases, the aqueous reaction mixture is in contact with an immiscible liquid that resides on top of the aqueous layer and therefore reduces evaporation of the aqueous layer. In some cases, this top layer immiscible liquid is less dense than the aqueous reaction mixture and therefore inherently adopts a top-layer position. In some cases, the aqueous reaction mixture is contained in a container (e.g., a tube, a well, or a pipette tip), where the container further contains the top-layer immiscible liquid.

In some cases, the aqueous reaction mixture is in contact with an immiscible liquid that resides below the aqueous layer. In some cases, this bottom-layer immiscible liquid is more dense than the aqueous reaction mixture and therefore inherently adopts a bottom-layer position. In some cases, the aqueous reaction mixture is contained in a container (e.g., a tube, a well, or a pipette tip), where the container further contains the bottom-layer immiscible liquid. In some cases, the aqueous reaction mixture is in contact with a first immiscible liquid that resides on top of the aqueous layer, and a second immiscible liquid that resides below the aqueous layer. Exemplary top- and/or bottom-layer immiscible liquids, compositions containing one or more of such immiscible liquids and an aqueous reaction mixture, and methods, systems, and articles of manufacture for forming, containing, and using such immiscible liquids and compositions are described in co-pending U.S. provisional application entitled “Methods and Compositions for Low Volume Liquid Handling,” U.S. Application No. 62/263,543, (filed on Dec. 4, 2015) the contents of which are incorporated by reference in the entirety.

In an exemplary embodiment, the aqueous reaction mixture contains additional salts, buffers, and solvents to provide for selective hybridization of bait oligonucleotides to target nucleic acid molecules. Such salts, buffers, and solvents include, but are not limited to SSC, SSPE, NaCl, Denhardt's Solution, bovine serum albumin, EDTA, Tween 20, and SDS. Additionally, or alternatively, the aqueous reaction mixture can contain components that accelerate the rate of hybridization and/or increase the selectivity of hybridization such as formamide, dextran sulphate, functionalized nanoparticles (e.g., functionalized carbon nanotubes), and tetramethylammonium chloride.

Additionally, or alternatively, the reaction mixture can contain one or more components that increase the thermal mass or heat transfer properties of the mixture. Such components, include but are not limited to, nanoparticles (e.g., metal nanoparticles such as nanoparticles of gold). In some embodiments, the nanoparticles (e.g., metal nanoparticles such as gold nanoparticles) are provided as a reaction mixture component as a colloidal nanoparticle solution. The colloidal solution (e.g., colloidal gold) is a suspension of sub-micrometer size particles of the metal in a fluid (e.g., water or an aqueous buffered solution such as PBS). In some embodiments, the nanoparticles are spherical, or a majority (e.g., >50%, or >95%) are spherical. In some embodiments, the nanoparticles are not spherical. The nanoparticles can be from about 1 nm in diameter to about 50 nm in diameter, from about 1 nm to about 25 nm, from about 1 nm to about 20 nm, from about 1 nm to about 15 nm, from about 1 nm to about 7 nm, from about 2 nm to about 20 nm, from about 2 nm to about 15 nm, from about 2 nm to about 10 nm, or about 1 nm, 2 nm, 3 nm, 3 nm, 4 nm, 5 nm, 7 nm, 10 nm, 13 nm, or 20 nm in diameter.

The nanoparticles (e.g., metal nanoparticles, such as gold nanoparticles) in the aqueous reaction mixture can be at a concentration of from about 0.1×10⁷ particles/μL to about 1×10⁸ particles/μL, from about 0.25×10⁷ particles/μL to about 7.5×10⁷ particles/μL, from about 0.5×10⁷ particles/μL to about 5×10⁷ particles/μL, from about 0.75×10⁷ particles/μL to about 4×10⁷ particles/μL, or from about 1×10⁷ particles/μL to about 3×10⁷ particles/μL. In some embodiments, the aqueous reaction mixture contains nanoparticles at a concentration of about 1.5×10⁷ particles/μL, 2×10⁷ particles/μL, 2.5×10⁷ particles/μL, 2.75×10⁷ particles/μL, 3×10⁷ particles/μL, 3.5×10⁷ particles/μL, or 4×10⁷ particles/μL.

In some embodiments, the aqueous reaction mixture contains tetramethy ammonium chloride at a concentration of from about 0.5 M to about 10 M, from about 0.75 M to about 8 M, from about 1 M to about 6 M, from about 1 M to about 4 M, from about 1.25 M to about 4 M, from about 1.5 M to about 4 M, from about 1 M to about 3 M, from about 1.25 to about 3 M, from about 1.5 M to about 3 M, from about 1.75 to about 3 M, from about 2 M to about 3 M, from about 2.25 M to about 3 M, from about 2.5 M to about 3 M, from about 2 M to about 2.75 M, from about 2.25 M to about 2.75 M, or from about 2.5 M to about 2.75 M. In some embodiments, the aqueous reaction mixture contains tetramethylammonium chloride at a concentration of about 1 M, 1.25 M, 1.5 M, 1.75 M, 2 M, 2.25 M, 2.5 M, 2.75 M, 3 M, 3.25 M, 3.5 M, 3.75 M, or 4 M.

In some embodiments, the aqueous reaction mixture contains colloidal gold at a concentration of from about 0.1×10⁷ gold particles/μL to about 1×10⁸ gold particles/μL, from about 0.25×10⁷ gold particles/μL to about 7.5×10⁷ gold particles/μL, from about 0.5×10⁷ gold particles/μL to about 5×10⁷ gold particles/μL, from about 0.75×10⁷ gold particles/μL to about 4×10⁷ gold particles/μL, or from about 1×10⁷ gold particles/μL to about 3×10⁷ gold particles/μL. In some embodiments, the aqueous reaction mixture contains colloidal gold at a concentration of about 1.5×10⁷ gold particles/μL, 2×10⁷ gold particles/μL, 2.5×10⁷ gold particles/μL, 2.75×10⁷ gold particles/μL, 3×10⁷ gold particles/μL, 3.5×10⁷ gold particles/μL, or 4×10⁷ gold particles/μL.

In some embodiments, the aqueous reaction mixture contains formamide at a concentration of from about 5% to about 40%, from about 5% to about 35%, from about 7.5% to about 40%, from about 7.5% to about 35%, from about 10% to about 35%, from about 10% to about 40%, from about 15% to about 40%, from about 15% to about 35%, from about 15% to about 30%, from about 15% to about 25%, from about 20% to about 25%, from about 10% to about 20%, or from about 15% to about 20%. In some embodiments, the aqueous reaction mixture contains formamide at a concentration of about 1%, 5%, 7.5%, 10%, 15%, 17%, 20%, 22%, 25%, 30%, 35%, or 40%.

In some embodiments, the aqueous reaction mixture contains dextran at a concentration of from about 0.1% to about 10%, from about 0.2% to about 7%, from about 0.25% to about 5%; from about 0.5% to about 5%, from about 0.5% to about 4%, from about 0.5% to about 3%, from about 0.5% to about 2.5%, from about 1% to about 4%, from about 1% to about 3.5%, from about 1% to about 3%, from about 1.25% to about 3.5%, from about 1.25% to about 3.25%, from about 1.25% to about 3%, from about 1.5% to about 3.5%, from about 1.5% to about 3.25%, or from about 1.5% to about 3%. In some embodiments, the aqueous reaction mixture contains dextran at a concentration of about 0.5%, 0.75%, 1%, 1.25%, 1.5%, 1.75%, 2%, 2.25%, 2.5%, 2.75%, 3%, 3.25%, 3.5%, or 4%.

In some embodiments, the aqueous reaction mixture contains SSPE buffer at a concentration of from about 0.1% to about 10%, from about 0.2% to about 8%, from about 0.25% to about 5%; from about 0.5% to about 5%, from about 0.5% to about 4%, from about 0.5% to about 3%, from about 0.5% to about 2.5%, from about 1% to about 4%, from about 1% to about 3.5%, from about 1% to about 3%, from about 1.25% to about 3.5%, from about 1.25% to about 3.25%, from about 1.25% to about 3%, from about 1.5% to about 3.5%, from about 1.5% to about 3.25%, or from about 1.5% to about 3%. In some embodiments, the aqueous reaction mixture contains SSPE buffer at a concentration of about 0.5%, 0.75%, 1%, 1.25%, 1.5%, 1.75%, 2%, 2.25%, 2.5%, 2.75%, 3%, 3.25%, 3.5%, or 4%.

In some embodiments, the aqueous reaction mixture contains Denhardt's Solution at a concentration of from about 0.1× to about 10×, from about 0.2× to about 8×, from about 0.25× to about 8×, from about 0.25× to about 5×; from about 0.5× to about 5×, from about 0.5× to about 4×, from about 0.5× to about 3×, from about 0.5× to about 2.5×, from about 1× to about 4×, from about 1× to about 3.5×, from about 1× to about 3×, from about 1× to about 2.5×, from about 1.25× to about 3.5×, from about 1.25× to about 3.25×, from about 1.25× to about 3×, from about 1.25× to about 2.5×, from about 1.5× to about 3.5×, from about 1.5× to about 3.25×, from about 1.5× to about 3×, from about 1.5× to about 2.5×, or from about 1.75× to about 2.5×. In some embodiments, the aqueous reaction mixture contains Denhardt's Solution at a concentration of about 0.5×, 0.75×, 1×, 1.25×, 1.5×, 1.75×, 2×, 2.25×, 2.5×, 2.75×, 3×, 3.25×, 3.5×, 4×, 4.5×, 5×, 5.5×, or 6×.

In some embodiments, the aqueous reaction mixture contains EDTA. As used herein, in the context of a reaction mixture that contains SSPE buffer and EDTA, it is understood that the separately recited EDTA refers to EDTA in addition to that provided in the SSPE buffer. In some embodiments, the aqueous reaction mixture contains EDTA at a concentration of from about 0.1 mM to about 50 mM, from about 0.2 mM to about 25 mM, from about 0.5 mM to about 15 mM; from about 0.5 mM to about 10 mM, from about 0.5 mM to about 8 mM, from about 0.5 mM to about 6 mM, from about 0.5 mM to about 4 mM, from about 0.5 mM to about 3 mM, from about 0.5 mM to about 2.5 mM, from about 1 mM to about 10 mM, from about 1 mM to about 7.5 mM, from about 1 mM to about 5 mM, from about 1 mM to about 4 mM, from about 1 mM to about 3 mM, from about 1 mM to about 2.5 mM, from about 1.5 mM to about 3 mM, from about 1.5 mM to about 3.5 mM, from about 1.5 mM to about 3.25 mM, or from about 1.5 mM to about 2.5 mM. In some embodiments, the aqueous reaction mixture contains EDTA at a concentration of about 0.1 mM, 0.25 mM, 0.75 mM, 1 mM, 1.25 mM, 1.5 mM, 1.75 mM, 2 mM, 2.25 mM, 2.5 mM, 2.75 mM, 3 mM, 3.25 mM, 3.5 mM, 3.75 mM, 4 mM, 8 mM, 10 mM, 15 mM, or 20 mM.

In some embodiments, the aqueous reaction mixture contains sodium dodecyl sulfate at a concentration of from about 0.001% to about 0.2%, from about 0.002% to about 0.1%, from about 0.005% to about 0.075%; from about 0.005% to about 0.08%, from about 0.005% to about 0.06%, from about 0.005% to about 0.04%, from 0.005% to about 0.02%, from about 0.01% to about 0.2%, from about 0.01% to about 0.1%, from about 0.01% to about 0.09%, from about 0.01% to about 0.08%, from about 0.01% to about 0.06%, from about 0.01% to about 0.04%, from about 0.01% to about 0.02%, from about 0.02% to about 0.2%, from about 0.02% to about 0.1%, from about 0.02% to about 0.09%, from about 0.02% to about 0.08%, from about 0.02% to about 0.07%, from about 0.02% to about 0.06%, from about 0.02% to about 0.04%, %, from about 0.03% to about 0.2%, from about 0.03% to about 0.1%, from about 0.03% to about 0.09%, from about 0.03% to about 0.08%, from about 0.03% to about 0.07%, from about 0.03% to about 0.06%, from about 0.03% to about 0.04%, or from about 0.03% to about 0.05%. In some embodiments, the aqueous reaction mixture contains sodium dodecyl sulfate at a concentration of about 0.005%, 0.01%, 0.02%, 0.03%, 0.04%, 0.05%, 0.06%, 0.07%, 0.08%, or 0.1%.

In some embodiments, the aqueous reaction mixture contains Tween 20 at a concentration of from about 0.0005% to about 0.01%, from about 0.0005% to about 0.005%, from about 0.0005% to about 0.0025%; from about 0.001% to about 0.01%, from about 0.001% to about 0.005%, from about 0.001% to about 0.003%, from 0.001% to about 0.0025%, or from about 0.0015% to about 0.0025%. In some embodiments, the aqueous reaction mixture contains Tween 20 at a concentration of about 0.0005%, 0.00075%, 0.001%, 0.0015%, 0.00175%, 0.002%, 0.0025%, 0.003%, 0.004%, or 0.005%.

### IV. Methods

Described herein are methods for enrichment of target nucleic acid molecules in a nucleic acid sample containing target and non-target nucleic acid molecules. The methods described herein utilize sequence specific hybridization between bait oligonucleotides and target nucleic acid molecules. In one aspect, the method includes: i) forming any one of the aqueous reaction mixtures described herein; ii) incubating the aqueous reaction mixture at a hybridization temperature of at least about 45° C. and no more than about 75° C., or at least about 50° C. and no more than about 70° C. for at least about 1 minute and less than about 1 hour to thereby hybridize at least a portion of the bait oligonucleotides to at least a portion of the target nucleic acid molecules; iii) after the incubating at the hybridization temperature, immobilizing at least a portion of the bait oligonucleotides to one or more solid surfaces; iv) removing at least a portion of the non-target nucleic acid molecules; and v) collecting enriched target nucleic acid molecules from the one or more solid surfaces, or amplification products thereof, thereby providing an enriched sample.

In another aspect, the method includes: (i) forming any one of the aqueous reaction mixtures described herein, ii) incubating the aqueous reaction mixture at a hybridization temperature of about 65° C. for at least about 10 minutes to thereby hybridize at least a portion of the bait oligonucleotides to at least a portion of the target nucleic acid molecules; and then, iii) immobilizing at least a portion of the bait oligonucleotides on one or more solid surfaces, thereby producing immobilized target nucleic acid molecule-bait oligonucleotide complexes; iv) separating at least a portion of the non-target nucleic acid molecules from the immobilized target nucleic acid molecule-bait oligonucleotide complexes; and v) recovering target nucleic acid molecules from the one or more solid surfaces, or amplification products thereof, thereby forming an enriched polynucleotide mixture of target and non-target nucleic acid molecules, wherein the polynucleotide mixture is enriched relative to the nucleic acid sample. In some cases, the method further includes vi) sequencing at least a portion of the nucleic acids in the enriched polynucleotide mixture, wherein an on-target rate of at least about 65% is achieved within a 10 minute incubation of the aqueous reaction mixture at the hybridization temperature.

In some cases, the method achieves an on-target rate of at least about 65% within about the first 10 minutes of incubation of the aqueous reaction mixture at the hybridization temperature. For example, incubating the aqueous reaction mixture at the hybridization temperature for 10 minutes and then performing steps iii)-vi) can result in an on-target rate as determined by high-throughput sequencing of at least about 65% (e.g., from about 65% to about 70%). As another example, incubating the aqueous reaction mixture at the hybridization temperature can result in an on-target rate as determined by high-throughput sequencing of at least about 65% (e.g., from about 65% to about 70%) if the incubating the aqueous reaction mixture at the hybridization temperature is performed for 10 minutes, a sample is then obtained from the aqueous reaction mixture, and the obtained sample is then analyzed by the steps of iii)-vi). The remaining portion of the aqueous reaction mixture can be incubated at the hybridization temperature for an additional period of time, e.g., 10, 20, 50, 70, 110, or 230, or more additional minutes, e.g. prior to performing the steps of iii)-iv) or iii)-v) on the remaining portion. As such, while an on-target rate of at least about 65% can be achieved with a 10 minute incubation at the hybridization temperature, and verified, the hybridization reaction can be performed for any length of time desired by one of ordinary skill in the art.

Similarly, in some cases, the method achieves an on-target rate of at least about 75% (e.g., from about 75% to about 80%) within about the first 30 minutes of incubation of the aqueous reaction mixture at the hybridization temperature. For example, incubating the aqueous reaction mixture at the hybridization temperature for 30 minutes and then performing steps iii)-vi) can result in an on-target rate as determined by high-throughput sequencing of at least about 75% (e.g., from about 75% to about 80%). Similarly, in some cases, the method achieves an on-target rate of at least about 80% (e.g., from about 75% to about 85%) within about the first 45 minutes, 60 minutes, 80 minutes, 90 minutes or 60-90 minutes of incubation of the aqueous reaction mixture at the hybridization temperature. For example, incubating the aqueous reaction mixture at the hybridization temperature for 45, 60, 80, 90, or 60-90 minutes and then performing steps iii)-vi) can, in some cases, result in an on-target rate as determined by high-throughput sequencing of at least about 80% (e.g., from about 65% to about 70%).

In some embodiments, the on-target rate is from about 65% to about 85%, or from about 66% to about 84%. In some embodiments, the on-target rate achieved with about 10 minutes of incubating the aqueous reaction mixture at the hybridization temperature is from about 65% to about 70%, from about 66% to about 69%, from about 66% to about 68%, or from about 66% to about 67%, or about 65%, 66%, 67%, 68%, 69%, or 70%. In some embodiments, the on-target rate achieved with about 20 minutes of incubating the aqueous reaction mixture at the hybridization temperature is from about 65% to about 70%, from about 66% to about 69%, from about 66% to about 68%, or from about 66% to about 67%, or about 65%, 66%, 67%, 68%, 69%, or 70%. In some embodiments, the on-target rate achieved with about 30 minutes of incubating the aqueous reaction mixture at the hybridization temperature is from about 75% to about 80%, from about 76% to about 79%, from about 76% to about 78%, or from about 76% to about 77%, or about 75%, 76%, 77%, 78%, 79%, or 80%. In some embodiments, the on-target rate achieved with about 240 minutes of incubating the aqueous reaction mixture at the hybridization temperature is from about 80% to about 85%, from about 81% to about 89%, from about 82% to about 89%, from about 83% to about 89%, or from about 83% to about 88%, or about 80%, 81%, 82%, 83%, 84%, or 85%.

In some embodiments, the target nucleic acid molecules of the enriched polynucleotide mixture comprise at least about 65% of total target and non-target nucleic acid molecules in the enriched polynucleotide mixture with less than about a 10 minute incubation of the aqueous reaction mixture at the hybridization temperature. For example, incubating the aqueous reaction mixture at the hybridization temperature for 10 minutes and then performing steps iii)-v) can result an enriched polynucleotide mixture wherein the target nucleic acid molecules of the enriched polynucleotide mixture comprise at least about 65% (e.g., from about 65% to about 70%) of total target and non-target nucleic acid molecules in the enriched polynucleotide mixture. As another example, incubating the aqueous reaction mixture at the hybridization temperature can result in an enriched polynucleotide mixture wherein target nucleic acid molecules comprise at least 65% (e.g., from about 65% to about 70%) of total target and non-target target nucleic acid molecules if the incubating the aqueous reaction mixture at the hybridization temperature is performed for 10 minutes, a sample is then obtained from the aqueous reaction mixture, and the obtained sample is then analyzed (e.g., by the steps of iii)-vi). The remaining portion of the aqueous reaction mixture can be incubated at the hybridization temperature for an additional period of time, e.g., 10, 20, 50, 70, 110, or 230, or more additional minutes, e.g. prior to performing the steps of iii)-iv) or iii)-v) on the remaining portion. As such, while an enriched polynucleotide mixture of at least 65% target nucleic acid molecules as a proportion of total target and non-target nucleic acid molecules can be achieved with a 10 minute incubation at the hybridization temperature, and verified, the hybridization reaction can be performed for any length of time desired by one of ordinary skill in the art.

Similarly, in some cases, the method achieves an enriched polynucleotide mixture of at least about 75% (e.g., from about 75% to about 80%) target nucleic acid molecules as a proportion of total target and non-target nucleic acid molecules within about the first 30 minutes of incubation of the aqueous reaction mixture at the hybridization temperature. Similarly, in some cases, the method achieves an enriched polynucleotide mixture of at least about 80% (e.g., from about 75% to about 85%) within about the first 45 minutes, 60 minutes, 80 minutes, 90 minutes or 60-90 minutes of incubation of the aqueous reaction mixture at the hybridization temperature. In some embodiments, the enriched polynucleotide mixture comprises from about 65% to about 85%, or from about 66% to about 84% target nucleic acid molecules as proportion of total target and non-target nucleic acid molecules. In some embodiments, the enriched polynucleotide mixture achieved with a 10 minute incubation at the hybridization temperature comprises from about 65% to about 70%, from about 66% to about 69%, from about 66% to about 68%, or from about 66% to about 67%, or about 65%, 66%, 67%, 68%, 69%, or 70% target nucleic acid molecules as proportion of total target and non-target nucleic acid molecules.

In some embodiments, the enriched polynucleotide mixture achieved with a 20 minute incubation at the hybridization temperature is from about from about 65% to about 70%, from about 66% to about 69%, from about 66% to about 68%, or from about 66% to about 67%, or about 65%, 66%, 67%, 68%, 69%, or 70%. In some embodiments, the enriched polynucleotide mixture achieved with a 30 minute incubation at the hybridization temperature is from about 75% to about 80%, from about 76% to about 79%, from about 76% to about 78%, or from about 76% to about 77%, or about 75%, 76%, 77%, 78%, 79%, or 80%. In some embodiments, the enriched polynucleotide mixture achieved with a 240 minute incubation at the hybridization temperature is from about 80% to about 85%, from about 81% to about 89%, from about 82% to about 89%, from about 83% to about 89%, or from about 83% to about 88%, or about 80%, 81%, 82%, 83%, 84%, or 85%.

The forming the aqueous reaction mixture can be performed by: i) forming an aqueous reaction pre-mixture comprising the nucleic acid sample, water, and bait oligonucleotides; ii) concentrating the aqueous reaction pre-mixture to a volume that is less than a total volume of the reaction mixture; and iii) contacting the aqueous reaction pre-mixture with a volume of hybridization buffer, wherein the volume of the hybridization buffer and the volume of the concentrated aqueous reaction pre-mixture prior to the contacting equals the total volume of the reaction mixture; and iv) denaturing the nucleic acid sample in the reaction pre-mixture by incubating the pre-mixture at a denaturing temperature and then cooling the nucleic acid to a hybridization temperature, thereby forming the aqueous reaction mixture.

The concentrating can be performed by any suitable method, such as subjecting the aqueous reaction pre-mixture to heat and/or vacuum. In some cases, the concentrating is performed by subjecting the aqueous reaction pre-mixture to heat and vacuum. In an exemplary embodiment, the concentrating is performed by subjecting the aqueous reaction pre-mixture to heat and vacuum in a centrifugal concentrator (e.g., SPEEDVAC®). Alternatively, the concentrating can be performed by isolating components of the aqueous reaction pre-mixture to a nucleic acid binding matrix and eluting the components in a smaller volume. In another alternative, the concentrating can be performed by diafiltration (e.g., using a MICROCON® concentrator). As yet another alternative, the concentrating can be performed by lyopholization. As yet another alternative, the concentrating can be performed by drying under a stream of inert gas, such as N₂ or argon.

In some cases, the incubating at the hybridization temperature is performed at a hybridization temperature of at least about 50° C. and no more than about 75° C.; at least about 55° C. and no more than about 75° C.; at least about 60° C. and no more than about 75° C.; at least about 65° C. and no more than about 75° C.; or at least about 65° C. and no more than about 70° C. In some cases, the incubating at the hybridization temperature is performed for at least about 2 minutes and no more than about 50 minutes; at least about 3 minutes and no more than about 45 minutes; at least about 5 minutes and no more than about 40 minutes; or at least about 10 minutes and no more than about 30 minutes. In some cases, the incubating at the hybridization temperature is performed for about 10, 20, 30, 45, 60, 80, 90, 120, 150, 180, 210, or 240 minutes.

In some cases, the incubating at the hybridization temperature is performed for at least about 2 minutes, at least about 10 minutes, at least about 20 minutes, at least about 30 minutes, at least about 40 minutes, at least about 50 minutes, at least about 60 minutes, at least about 70 minutes, at least about 80 minutes, at least about 90 minutes, at least about 100 minutes, at least about 120 minutes, at least about 150 minutes, at least about 180 minutes, at least about 210 minutes, or at least about 240 minutes. In some cases, the incubating at the hybridization temperature is performed for at least about 2 minutes and no more than 4 hours, at least about 2 minutes and no more than 3 hours, at least about 2 minutes and no more than 1.5 hours, at least about 2 minutes and no more than 1 hour, at least about 2 minutes and no more than 45 minutes, or at least about 2 minutes and no more than about 30 minutes. In some cases, the incubating at the hybridization temperature is performed for at least about 10 minutes and no more than 4 hours, at least about 10 minutes and no more than 3 hours, at least about 10 minutes and no more than 2.5 hours, at least about 10 minutes and no more than 2 hours, at least about 10 minutes and no more than 1.5 hours, or at least about 10 minutes and no more than 1 hour, at least about 10 minutes and no more than 45 minutes, or at least about 10 minutes and no more than about 30 minutes.

In some cases, the incubating at the hybridization temperature is performed for at least about 30 minutes and no more than 4 hours, at least about 30 minutes and no more than 3.5 hours, at least about 30 minutes and no more than 3 hours, at least about 30 minutes and no more than 2.5 hours, at least about 30 minutes and no more than 2 hours, at least about 30 minutes and no more than 1.5. hours, or at least about 30 minutes and less than about 1 hours. In some cases, the incubating at the hybridization temperature is performed for at least about 45 minutes and no more than 4 hours, at least about 45 minutes and no more than 3.5 hours, at least about 45 minutes and no more than 3 hours, at least about 45 minutes and no more than 2.5 hours, at least about 45 minutes and no more than 2 hours, at least about 45 minutes and no more than 1.5 hours, or at least about 45 minutes and less than about 1 hour. In some cases, the incubating at the hybridization temperature is performed for at least about 60 minutes and no more than 4 hours, at least about 60 minutes and no more than 3.5 hours, at least about 60 minutes and no more than 3 hours, at least about 60 minutes and no more than 2.5 hours, at least about 60 minutes and no more than 2 hours, or at least about 60 minutes and less than about 1.5 hours. In some cases, the incubating at the hybridization temperature is performed for at least about 90 minutes and no more than 4 hours, at least about 90 minutes and no more than 3.5 hours, at least about 90 minutes and no more than 3 hours, at least about 90 minutes and no more than 2.5 hours, or at least about 90 minutes and no more than 2 hours.

The denaturing can be performed by incubating the aqueous reaction pre-mixture at a temperature of at least about 85° C. For example, the denaturing can be performed by incubating the aqueous reaction pre-mixture at a temperature of at least about 85° C. and no more than about 100° C.; at least about 90° C. and no more than about 100° C.; at least about 90° C. and no more than about 99° C.; or at least about 95° C. and no more than about 99° C. In some cases, the denaturing is performed by incubating the aqueous reaction pre-mixture at a temperature of about 90° C., 95° C., or 98° C.

The denaturing can be performed for at least about 0.5 minutes. In some cases, the denaturing is performed for at least about 1 minute and no more than about 30 minutes; at least about 5 minutes and no more than 20 minutes; or at least about 5 minutes and no more than 15 minutes. In some cases, the denaturing is performed for about 10 minutes (e.g., at 95° C.). Extended denaturing can be undesirable due to nucleic acid hydrolysis, evaporation, and the like.

After denaturing and hybridizing target nucleic acid molecules to bait oligonucleotides, at least a portion of the bait oligonucleotides can be immobilized to one or more solid surfaces, thereby immobilizing at least a portion of target nucleic acid molecules to the solid surfaces. In an exemplary embodiment, the solid surfaces are beads (e.g., magnetic beads). For example, the solid surfaces can be avidin or streptavidin-coated beads that can capture biotinylated bait oligonucleotides. Immobilization is performed by contacting the aqueous reaction mixture to the one or more solid surfaces and incubating the composition for a time and temperature sufficient to cause immobilization.

Immobilization can be performed for at least about 1 minute and no more than overnight. In some cases, immobilization is performed for at least 5 minutes and not more than 12 hours; at least 10 minutes and not more than 8 hours; at least 15 minutes and not more than 4 hours; at least 20 minutes and not more than 2 hours; at least 30 minutes and not more than 1 hour, at least about 30 minutes at not more than overnight, at least about 30 minutes and not more than 12 hours, at least about 30 minutes and not more than 8 hours, or at least about 30 minutes and not more than about 4 hours. In some cases, immobilization is performed for about 10 minutes, about 20 minutes, about 30 minutes, about 45 minutes, or about 1 hour. Immobilization can be performed at a temperature of about of about 25° C., 30° C., 37° C., 40° C., 45° C., 50° C., 55° C., 60° C., 65° C., or 70° C. Immobilization can be performed at a temperature of about of at least about 40° C. and not more than about 75° C.; at least about 45° C. and not more than about 70° C.; at least about 50° C. and not more than about 70° C.; at least about 55° C. and not more than about 70° C.; or at least about 60° C. and not more than about 70° C. In some cases, immobilization is performed at about 65° C. For example, immobilization can be performed at about 65° C. for about 45 minutes. As another example, immobilization can be performed at about 65° C. for at least about 45 minutes.

After immobilization, the non-target nucleic acids that are not immobilized onto the one or more solid surfaces can be removed. Such removal can be performed by separating the aqueous layer of the aqueous reaction mixture from the one or more solid surfaces. In some cases, the separating is performed by pipetting the aqueous reaction mixture from the solid surfaces. In some cases, the separating is performed by filtering the solid surfaces out of the aqueous reaction mixture, e.g., with a 0.2 μm spin filter. In an exemplary embodiment, the solid surfaces are magnetic beads, and the removing is performed by attracting the magnetic beads to a magnet applied to one surface of a container containing the aqueous reaction mixture, and pipetting away the bulk liquid.

The removing can be performed in multiple steps. For example, the solid surfaces can be washed with various hybridization wash buffers at selected stringencies to remove non-specifically hybridized non-target nucleic acid molecules. In some cases, the wash buffers are heated to, or to about, the hybridization temperature. In some cases, the wash buffers are at room temperature. In some cases, the solid surfaces are washed with wash buffers at the hybridization temperature and wash buffers at room temperature. In some cases, wash steps can include an incubation period of from 1 to about 10 minutes, or about 5 minutes.

After removing non-target nucleic acid molecules, the enriched target nucleic acid molecules, or amplification products thereof, can be collected. In some cases, the collecting is performed by eluting the enriched target nucleic acid molecules from the immobilized bait oligonucleotides. In some cases, the collecting is performed by digesting the bait oligonucleotide RNA present in an RNA:DNA hybrid with target nucleic acid molecules. In some cases, the collecting is performed by cleaving (e.g., via chemical or enzymatic means) a region within the bait oligonucleotides (e.g., a linker between the bait and the label) to release hybridized target nucleic acids from the solid surfaces.

In some cases, the collecting is performed by amplifying immobilized target nucleic acid molecules. For example, one or more universal primers that are complementary to adaptor sequences can be used to amplify (e.g., by PCR) the solid surface immobilized target nucleic acid molecules, producing amplicons in the aqueous reaction mixture. The aqueous reaction mixture can then be harvested from the solid surfaces to collect amplification products of the target nucleic acid molecules.

In some cases, the target nucleic acid molecules of the enriched sample comprise at least about 50% of the total target and non-target nucleic acid molecules in the enriched sample. In some cases, the target nucleic acid molecules of the enriched sample comprise at least about 50% and no more than about 95%; at least about 50% and no more than about 90%; at least about 55% and no more than about 85%; at least about 60% and no more than about 80%; or about 65%, 70%, or 75% of the total target and non-target nucleic acid molecules in the enriched sample.

In some cases, the method provides a median enrichment of target nucleic acid molecules or baited region in the sample (relative to a sample that is not enriched or relative the genome) of at least 100-fold, at least 150-fold, at least 200-fold, at least 250-fold, at least 300-fold, at least 350-fold, at least 400-fold, at least 500-fold, at least 750-fold, at least 1,000-fold, or more. In some cases, the method provides a median enrichment of target nucleic acid molecules or baited region in the sample (relative to a sample that is not enriched or relative the genome) of at least about 100-fold and no more than 10,000-fold; at least about 200-fold and no more than 5,000-fold; or at least about 500-fold and no more than 2,500-fold.

### V. Examples

### Example 1: Hybrid Capture

This experiment demonstrates the high on-target rate achievable in a hybrid capture reaction with a short hybridization time and a high concentration of bait oligonucleotides. The experiment further demonstrates that the method does not introduce substantially more G/C or A/T bias as compared to commercially available hybrid capture reagents, kits, and methods.

The following reagents were combined in a reaction chamber and concentrated to dryness in a centrifugal vacuum concentrator thereby providing an aqueous reaction pre-mixture:

i) 2,400 ng of an Illumina P5 and P7 adaptor-ligated library of genomic nucleic acid fragments;

ii) 0.75 pmol each of Illumina P5 and P7 blocking oligonucleotides;

iii) 5 μg human Cₒt1 DNA; and

iv) 1.2 pmol XGEN® lockdown probe pool of biotinylated bait oligonucleotides.

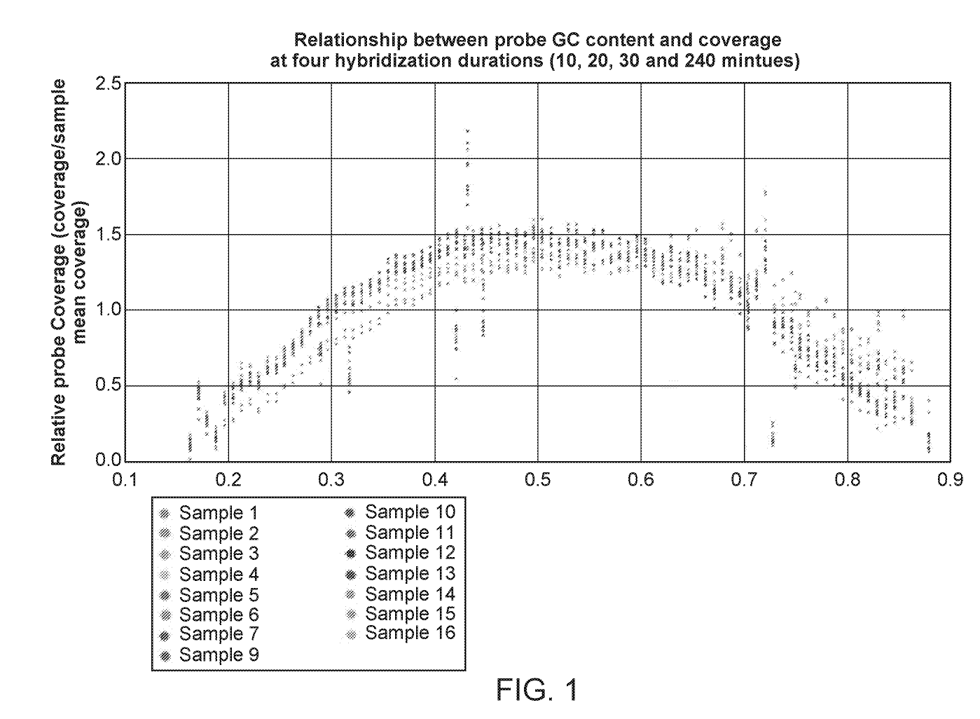

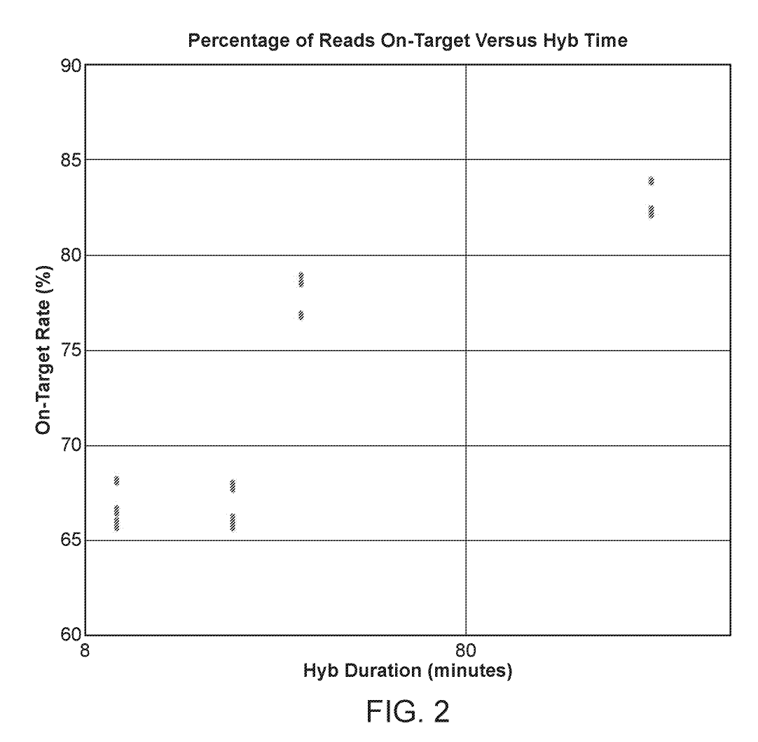

The results depicted in FIG. 1 were generated using the XGEN® pan cancer panel bait oligonucleotides (IDT), and the results depicted in FIG. 2 were generated using the XGEN® exome panel bait oligonucleotides (IDT).

The concentrated reaction pre-mixture was re-suspended to a total volume of 2 μL with an aqueous hybridization solution. The reaction pre-mixture was mixed thoroughly to ensure re-suspension of nucleic acid components. The reaction pre-mixture was then incubated at 95° C. for 10 minutes to denature the library of genomic nucleic acid fragments, and cooled to a hybridization temperature of about 65° C., thereby providing the hybrid capture reaction mixture. The hybrid capture reaction mixture was then incubated at 65° C. for 10-240 minutes to capture target nucleic acids by hybridization to the bait oligonucleotides. Samples 1-4 were incubated for 10 minutes to capture target nucleic acids by hybridization to the bait oligonucleotides. Samples 6-8 were incubated for 20 minutes to capture target nucleic acids by hybridization to the bait oligonucleotides. Samples 9-12 were incubated for 30 minutes to capture target nucleic acids by hybridization to the bait oligonucleotides. Samples 13-16 were incubated for 240 minutes to capture target nucleic acids by hybridization to the bait oligonucleotides.

The bait oligonucleotides were then captured by combining the reaction mixture with streptavidin magnetic beads, and incubating at 65° C. for an additional 5 minutes with occasional mixing, thereby immobilizing the bait oligonucleotides and captured target nucleic acids. After immobilization, a low stringency wash buffer was added to the reaction mixture at room temperature with mixing, the beads were separated from the bulk solution with a magnet, and the was buffer was removed. A high stringency heated wash buffer was then added to the beads with mixing. The beads and heated wash buffer were incubated at 65° C. for 5 minutes, the beads were separated from the bulk of the solution with a magnet, and the wash buffer was removed. Heated wash buffer was again added to the reaction mixture with mixing. The reaction mixture was incubated at 65° C. for an additional 5 minutes, the beads were separated from the bulk of the solution with a magnet, and the wash buffer was removed. This high-stringency wash was repeated four more times for a total of six wash cycles. After was buffer was removed from the final high-stringency wash cycle, the beads were re-suspended in nuclease free water, thereby providing beads containing immobilized bait oligonucleotides hybridized to an enriched fraction of target nucleic acid molecules.

The enriched fraction of target nucleic acid molecules was amplified in an amplification reaction mixture using universal PCR primers that amplify adaptor ligated nucleic acid molecules. The amplicons were produced as non-immobilized nucleic acid molecules in the reaction mixture, isolated, and subjected to high-throughput sequencing to assess the level of enrichment, identify genetic variants, assess cancer risk, and other screening procedures. High-throughput sequencing results are provided in FIGS. 1 and 2.

As shown in FIG. 1, hybrid capture with 1.2 pmol of the XGEN pan cancer panel (IDT) in a 2 μL reaction for 10, 20, 30, and 240 minutes does not exhibit a significant hybridization time-dependent G/C or A/T bias in the enriched sample. As shown in FIG. 2, the on-target rate for a sample prepared by hybrid capture with 1.2 pmol of the XGEN exome panel (IDT) in a 2 μL reaction is greater than 65% after a 10 or 20 minute hybridization time, over 75% after a 30 minute incubation time, and over 80% after a 240 minute hybridization time.

### Example 2: Hybrid Capture with Individually Synthesized 5′-Biotinylated DNA Oligonucleotide Probes

This experiment demonstrates hybrid capture with a pooled sample of 12 different adapter ligated nucleic acid samples.

### Reagents:

The following stock reagents were made or provided:

| | |
|---|---|
| Saline-Sodium | 2 mM sodium phosphate, pH 7.4; 30 mM |
| Phosphate-EDTA (SSPE) | sodium chloride, 0.2 mM EDTA |
| buffer 20X | |
| Dextran 50% | 50 g Dextran in 100 mL water |
| Denhardt's Solution 50X | 1% Ficoll (type 400), 1% |
| | polyvinylpyrrolidone, and 1% bovine serum |
| | albumin |
| EDTA 0.5M | 0.5 moles EDTA in 1 L of water |
| SDS 20% | 20 g SDS in 100 mL water |
| Tween 20, 99% | |
| NaCl 5M | 5 moles of sodium chloride in 1 L of water |
| Tris HCl 1M | 1 mole of Tris-HCl in 1 L of water |

Stock reagents above were combined to produce Hyb buffer containing 2% Dextran, 4% SSPE buffer, 4×Denhardt's Solution, 4 mM EDTA (in addition to EDTA from SSPE buffer), 0.08% SDS, 0.004% Tween 20. Stock reagents above were combined to produce binding buffer containing 1 M NaCl, 10 mM Tris HCl, 1 mM EDTA, and 0.01% Tween 20.

The following reagents were combined:

| | | |
|---|---|---|
| Pooled Sample DNA (n = 1-12) | 200-2,400 | ng |
| C₀t 1 DNA (10 mg/mL) | 5 | μl |
| Illumina P7 Blocking Oligonucleotides (IDT) | 1 | μL |
| Illumina P5 Blocking Oligonucleotides (IDT) | 1 | μL |
| xGen-lockdown Probe Mix (IDT) | 4 | μL |
| Hyb Buffer | 1 | μL |

The reagents were combined as follows in a well of a microwell plate. Approximately 80-200 ng of P5 and P7 adapter ligated library for each sample were combined into a single well of a hybrid capture plate. 12 samples were pooled (˜2,400 ng). 12 μL of hybridization master mix (i.e., 5 μL C₀t 1 DNA, 1 μL P5 and 1 μL P7 blocking oligonucleotides, 4 μL xGen-lockdown Probe Mix, and 1 μL Hyb buffer were introduced into the well. The well was dried under vacuum at a temperature of less than 70° C.

After the wells were totally dry, the reagents in the well were re-suspended in 2 μL of elution solution (1.2 μL of 5M tetramethylammonium chloride, 0.32 μL, of formamide (100%), and water to a final volume of 2 μL). 10 μL, of vapor lock was added to the well to prevent evaporation and provide additional thermal mass, thereby increasing the thermal stability of the reaction. The sample was then incubated under hybrid capture conditions as follows: denature at 95° C. for 5 minutes, hybridize at 65° C. for approximately 90 minutes.

25 μL of streptavidin-coated magnetic beads (Dynabeads, Thermo Fisher M-270) were added to a clean well in a separate microwell plate. The beads were washed by pipetting 100 μL of binding buffer into the well and mixing for 30 seconds. Binding buffer was removed by placing the plate on a magnet and removing the supernatant. The wash was repeated three times, and the beads were re-suspended in 25 μL of binding buffer.

The following wash buffer stock reagents were made (to be used at 1×):

| | |
|---|---|
| 10x Wash 1 | 20 X SSC, 1% SDS |
| 10x Wash 2 | 10 X SSC, 1% SDS |
| 10x Wash 3 | 1 mM NaCl, 100 mM Tris-Cl, pH 8.5, 10 mM |
| | EDTA |
| 10x Stringent Wash | 1.5X SSC, 1% SDS |

After the hybridization was completed, the streptavidin-coated magnetic beads were mixed for 30 seconds to re-suspend, and the 25 μL were added to the hybridization reaction and mixed for 2 minutes by pipetting up and down. Then, 100 μL of wash buffer 1 at a temperature of about 70° C. was added to the bead/DNA reaction and mixed for 15 seconds. The plate was then placed on a magnet and the supernatant was removed. Then 200 μL of stringent wash buffer at a temperature of about 70° C. was added to the bead/DNA reaction and mixed for 30 seconds, followed by a 2.5 minute incubation at 65° C. The plate was then placed on a magnet and the supernatant was removed. The heated stringent wash, 2.5 minute incubation, magnetic capture, and removal of supernatant was repeated. The beads were then washed by: (i) adding 200 μL of room temperature wash buffer 1 to the well, pipetting to mix for 30 seconds, placing the plate on a magnet, and removing the supernatant; (ii) adding 200 μL of room temperature wash buffer 2 to the well, pipetting to mix for 30 seconds, placing the plate on a magnet, and removing the supernatant; and (iii) adding 200 μL of room temperature wash buffer 3 to the well, pipetting to mix for 30 seconds, placing the plate on a magnet, and recovering the supernatant.

Polynucleotides captured by the hybrid capture reaction were amplified directly from the beads by incubating the streptavidin beads bound to the target nucleic acid: bait oligonucleotides in 1×PCR mix (25 μL Kapa HiFi 2× polymerase, 5 μL primer, 20 μL water), and performing PCR amplification with P5 and P7 adapter-specific amplification primers.

### Example 3: Hybrid Capture with Colloidal Gold

This experiment demonstrates hybrid capture using different elution solutions for the hybridization step in the presence or absence of colloidal gold.

### Reagents:

The following stock reagents were made:

| | |
|---|---|
| Saline-Sodium | 2 mM sodium phosphate, pH 7.4; 30 mM |
| Phosphate-EDTA (SSPE) | sodium chloride, 0.2 mM EDTA |
| buffer 20X | |
| Dextran 50% | 50 g Dextran in 100 mL water |
| Denhardt's Solution 50X | 1% Ficoll (type 400), 1% |
| | polyvinylpyrrolidone, and 1% bovine serum |
| | albumin |
| EDTA 0.5M | 0.5 moles EDTA in 1 L of water |
| SDS 20% | 20 g SDS in 100 mL water |
| Tween 20, 99% | |
| NaCl 5M | 5 moles of sodium chloride in 1 L of water |
| Tris HCl 1M | 1 mole of Tris-HCl in 1 L of water |
| Tetramethyl Ammonium | 5 moles/L in water (available from Sigma- |
| Chloride (TMAC) 5M | Aldrich under product number T3411) |
| Formamide ≧99.5% | available from Sigma-Aldrich under product |
| | number F9037 |
| Colloidal Gold (5 nm | available from Sigma-Aldrich under product |
| diameter, OD 1, stabilized | number 752568 |
| suspension in 0.1 mM PBS; | |
| approximately 5.5 × 10¹³ | |
| particles/mL) | |

Stock reagents above were combined to produce Hyb buffer containing 2% Dextran, 4% SSPE buffer, 4×Denhardt's Solution, 4 mM EDTA (in addition to EDTA from SSPE buffer), 0.08% SDS, 0.004% Tween 20. Stock reagents above were combined to produce binding buffer containing 1 M NaCl, 10 mM Tris HCl, 1 mM EDTA, and 0.01% Tween 20.

The following reagents were combined:

| | | |
|---|---|---|
| Pooled Sample DNA (n = 1-12) | 200-2,400 | ng |
| C₀t 1 DNA (10 mg/mL) | 5 | μl |
| Illumina P7 Blocking Oligonucleotides (IDT) | 1 | μL |
| Illumina P5 Blocking Oligonucleotides (IDT) | 1 | μL |
| xGen-lockdown Probe Mix (IDT) | 4 | μL |
| Hyb Buffer | 1 | μL |

The reagents were combined as follows in a well of a microwell plate. Approximately 80-200 ng of P5 and P7 adapter ligated library for each sample were combined into a single well of a hybrid capture plate. 12 samples were pooled (˜2,400 ng). 12 μL of hybridization master mix (i.e., 5 μL C₀t 1 DNA, 1 μL P5 and 1 μL P7 blocking oligonucleotides, 4 μL xGen-lockdown Probe Mix, and 1 μL Hyb buffer) were introduced into the well. The well was dried under vacuum at a temperature of less than 70° C.

After the wells was totally dry, the reagents in the well were re-suspended in 2 μL of elution solution. Two different elution solutions were compared: version 1.1 (V1.1), which contained 1 μL of 5M tetramethylammonium chloride, 0.4 μL, of formamide ≧99.5%, and water to a final volume of 2 μL; and version 1.2 (V1.2), which contained 1.1 μL, of 5M tetramethylammonium chloride, 0.4 μL of formamide ≧99.5%, and 0.5 μL, of colloidal gold (approximately 2.75×10⁷ 5 nm gold particles) for a final volume of 2 μL. 10 μL of vapor lock was added to the well to prevent evaporation and add thermal mass, thereby increasing the thermal stability of the reaction. The sample was then incubated under hybrid capture conditions as follows: denature at 95° C. for 5 minutes, hybridize at 65° C. for approximately 90 minutes.

25 μL, of magnetic streptavidin beads (Dynabeads, Thermo Fisher M-270) were added to a clean well in a separate microwell plate. The beads were washed by pipetting 100 μL, of binding buffer into the well and mixing for 30 seconds. Binding buffer was removed by placing the plate on a magnet and removing the supernatant. The wash was repeated three times, and the beads were re-suspended in 25 μL, of binding buffer.

The following wash buffer stock reagents were made (to be used at 1×):

| | |
|---|---|
| 10x Wash 1 | 20 X SSC, 1% SDS |
| 10x Wash 2 | 10 X SSC, 1% SDS |
| 10x Wash 3 | 1 mM NaCl, 100 mM Tris-Cl, pH 8.5, 10 mM |
| | EDTA |
| 10x Stringent Wash | 1.5X SSC, 1% SDS |

After the hybridization was completed, the streptavidin beads were mixed for 30 seconds to re-suspend, and the 25 μL were added to the hybridization reaction and mixed for 2 minutes by pipetting up and down. Then, 100 μL of wash buffer 1 at a temperature of about 70° C. was added to the bead/DNA reaction and mixed for 15 seconds. The plate was then placed on a magnet and the supernatant was removed. Then 200 μL of stringent wash buffer at a temperature of about 70° C. was added to the bead/DNA reaction and mixed for 30 seconds, followed by a 2.5 minute incubation at 65° C. The plate was then placed on a magnet and the supernatant was removed. The heated stringent wash, 2.5 minute incubation, magnetic capture, and removal of supernatant was repeated. The beads were then washed by: (i) adding 200 μL of room temperature wash buffer 1 to the well, pipetting to mix for 30 seconds, placing the plate on a magnet, and removing the supernatant; (ii) adding 200 μL of room temperature wash buffer 2 to the well, pipetting to mix for 30 seconds, placing the plate on a magnet, and removing the supernatant; and (iii) adding 200 μL of room temperature wash buffer 3 to the well, pipetting to mix for 30 seconds, placing the plate on a magnet, and recovering the supernatant.

Polynucleotides captured by the hybrid capture reaction were amplified directly from the beads by incubating streptavidin beads bound to the target nucleic acid: bait oligonucleotides in 1×PCR mix (25 μL Kapa HiFi 2× polymerase, 5 μL primer, 20 μL water), and performing PCR amplification with P5 and P7 adapter-specific amplification primers.

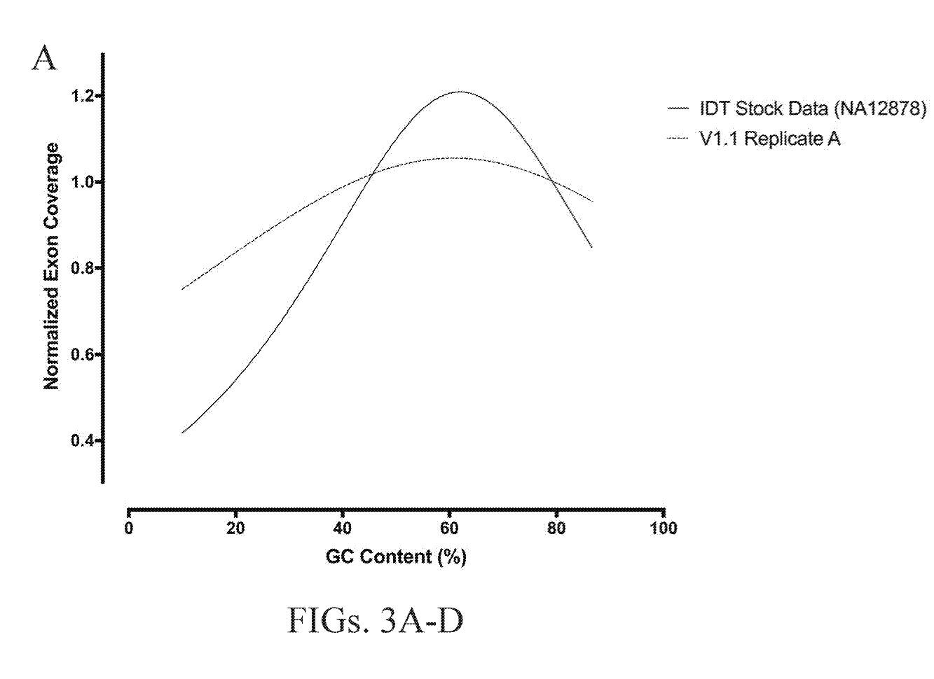

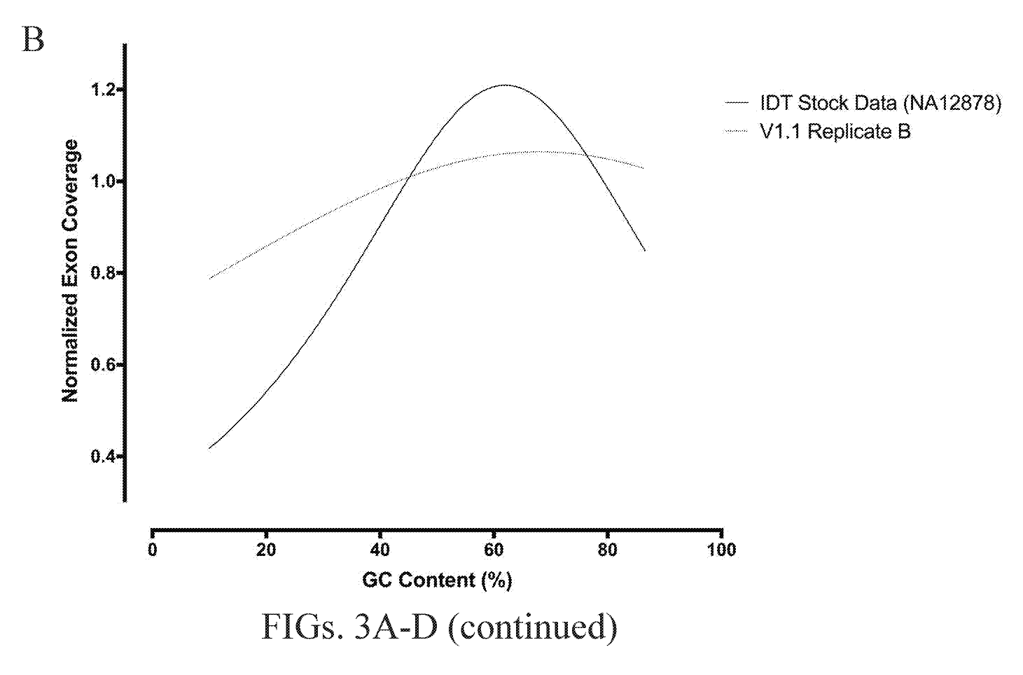

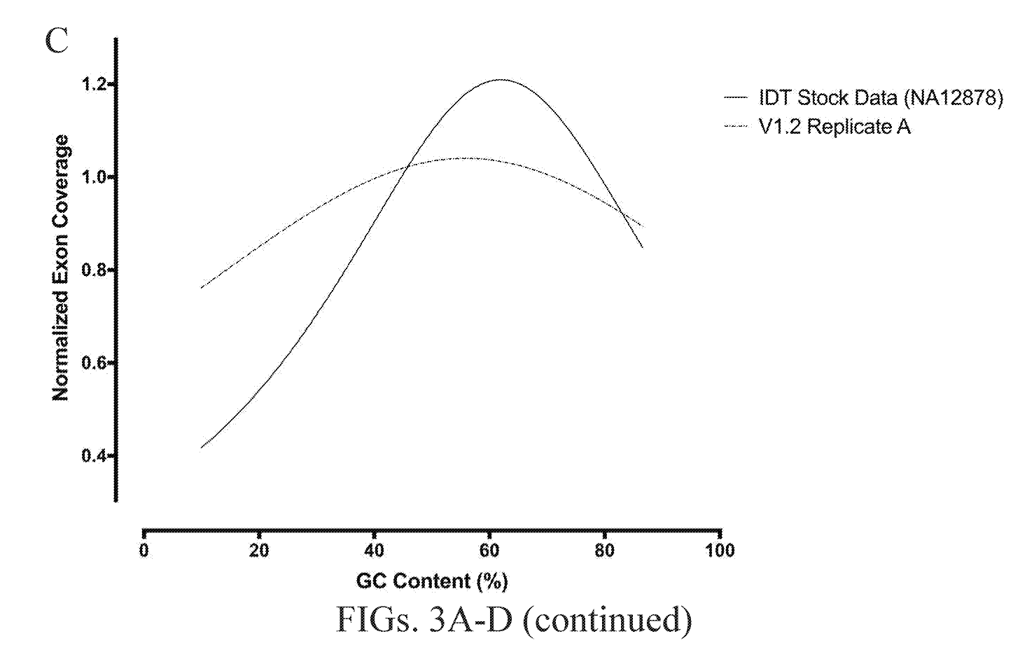

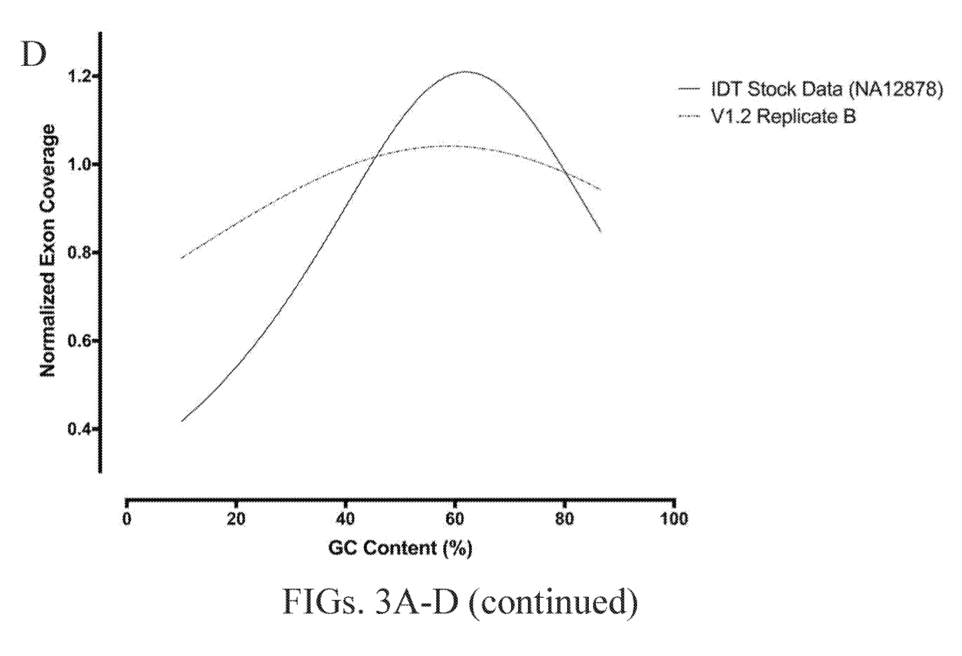

The amplicons were produced as non-immobilized nucleic acid molecules in the reaction mixture, isolated, and subjected to high-throughput sequencing to assess the level of enrichment. The protocol is performed in duplicate (Replicate A and B) using the two different elution solutions (V1.1 and 1.2) for a total of four datasets. The datasets were independently analyzed to calculate normalized exon coverage (dependent variable) versus % GC content (independent variable), and fit to a Lorentzian curve using default parameters in GraphPad Prism V7.0a. The results were compared with high-throughput sequencing data provided by the manufacturer and generated according to the manufacturer's protocol. The results are illustrated in FIGS. 3A-D, for each indicated elution solution (V1.1 or 1.2) and replicate (A and B), and compared with high-throughput sequencing data provided by the manufacturer and generated according to the manufacturer's protocol. As shown in FIGS. 3A-D, hybrid capture with V1.1 and V1.2 elution solutions provides a high degree of uniformity in exon capture over a wide range of different GC content as compared to the manufacturer's protocol.

An area under the curve analysis of the data shown in FIGS. 3A-D was performed using GraphPad Prism V7.0a, using the following parameters: Baseline=Y=1; Minimum Peak Height=Ignore any peak that is less than 10% of the distance from minimum to maximum Y; Minimum Peak Width=Ignore any peak that is defined by fewer than 5 adjacent points; Peak Direction=also consider peaks that go below baseline; and Significant Digits=show 4 significant digits. The results are shown in the table below.

| | V1.2 | V1.1 | V1.2 | V1.1 | IDT Stock |
|---|---|---|---|---|---|
| | Replicate | Replicate | Replicate | Replicate | Data |
| Column 1 | A | A | B | B | (NA12878) |
| Baseline | 1 | 1 | 1 | 1 | 1 |
| Area of | 0.6063 | 0.6645 | 0.679 | 1.15 | 0.475 |
| Positive | | | | | |
| Peaks | | | | | |
| Area of | 2.384 | 2.317 | 2.298 | 0.1524 | 19.09 |
| Negative | | | | | |
| Peaks | | | | | |
| Total Area | 8.435 | 8.804 | 8.403 | 8.726 | 43.63 |
| Net Area | −1.778 | −1.653 | −1.619 | 0.9972 | −18.61 |
| Total Peak | 2.99 | 2.982 | 2.977 | 1.302 | 19.56 |
| Area | | | | | |
| Number | 7 | 6 | 7 | 9 | 6 |
| of Peaks | | | | | |

As illustrated in the table above, the manufacturer's protocol produces a coverage profile across the GC content range that is much wider (less uniform) that the methods described herein. For example, the “Total Peak Area” measurement shows that the methods described herein (Total Peak Area=1.30−2.99) can perform an order of magnitude more uniformly than the manufacturer's (Total Peak Area=19.56).

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope. References and citations to other documents, such as patents, patent applications, patent publications, journals, books, papers, and web contents throughout this disclosure are hereby incorporated herein by reference in their entirety for all purposes.

What is claimed is:

1. An aqueous reaction mixture for enrichment of target nucleic acid molecules from a nucleic acid sample comprising a plurality of target nucleic acid molecules and a plurality of non-target nucleic acid molecules, the reaction mixture comprising:
a) a plurality of structurally distinct bait oligonucleotides, wherein the bait oligonucleotides comprise sequences complementary to the plurality of target nucleic acid molecules;
b) the plurality of target nucleic acids;
c) the plurality of non-target nucleic acids; and
d) water,
wherein the concentration of bait oligonucleotides in the aqueous reaction mixture is at least 0.5 pmol/μL.

2. The aqueous reaction mixture of claim 1, wherein the concentration of bait oligonucleotides in the aqueous reaction mixture is from about 0.6 pmol/μL to about 2 pmol/μL.

3. The aqueous reaction mixture of claim 1 or 2, wherein the total concentration of target and non-target nucleic acid molecules in the aqueous reaction mixture is at least about 50 ng/μL.

4. The aqueous reaction mixture of claim 3, wherein the total concentration of target and non-target nucleic acid molecules is from about 150 ng/μL to about 300 ng/μL.

5. The aqueous reaction mixture of claim 3, wherein the total concentration of target and non-target nucleic acids in the aqueous reaction mixture is 250 ng/μL.

6. The aqueous reaction mixture of claim 3, wherein the total concentration of target and non-target nucleic acids in the aqueous reaction mixture is from about 100 ng/μL to about 1,500 ng/μL.

7. The aqueous reaction mixture of claim 3, wherein the total concentration of target and non-target nucleic acids in the aqueous reaction mixture is 500 ng/μL.

8. The aqueous reaction mixture of claim any one of the preceding claims, wherein the aqueous reaction mixture has a volume of from about 1 μL to 5 μL.

9. The aqueous reaction mixture of claim 8, wherein the aqueous reaction mixture has a volume of about 2 μL.

10. The aqueous reaction mixture of any one of the preceding claims, wherein the target nucleic acid molecules and non-target nucleic acid molecules consist of a library of adaptor ligated nucleic acid fragments.

11. The aqueous reaction mixture of claim 10, wherein the library of adaptor ligated nucleic acid fragments is a library of adaptor ligated genomic DNA fragments.

12. The aqueous reaction mixture of claim 10 or 11, wherein the aqueous reaction mixture further comprises blocking oligonucleotides, wherein the blocking oligonucleotides are complementary to one or more adaptors of the adaptor ligated nucleic acid fragments.

13. The aqueous reaction mixture of any preceding claim, wherein the aqueous reaction mixture further comprises a blocking nucleic acid, wherein the blocking nucleic acid hybridizes to repetitive sequences in at least a portion of the non-target nucleic acid molecules.

14. The aqueous reaction mixture of claim 13, wherein the library of adaptor ligated nucleic acid fragments is a library of adaptor ligated genomic DNA fragments, and the blocking nucleic acid is Cₒt1-DNA, Cₒt2-DNA, or Cₒt3-DNA, or a mixture of two or more of the foregoing.

15. The aqueous reaction mixture of any one of the preceding claims, wherein the bait oligonucleotides comprise RNA oligonucleotides.

16. The aqueous reaction mixture of any one of the preceding claims, wherein the label of the bait oligonucleotides comprises biotin.

17. The aqueous reaction mixture of any one of the preceding claims, wherein the reaction mixture comprises:
i) tetramethylammonium chloride at a concentration of from about 1 M to about 4 M;
ii); colloidal gold at a concentration of from about 0.5×10⁷ gold particles/μL to about 5×10⁷ gold particles/μL;
iii) formamide at a concentration of from about 5% to about 35%;
iv) dextran at a concentration of from about 0.25% to about 5%;
v) SSPE buffer at a concentration of from about 0.2% to about 8%;
vi) Denhardt's Solution at a concentration of from about 0.25× to about 8×;
vii) EDTA at a concentration of from about 0.25 mM to about 50 mM;
viii) sodium dodecyl sulfate at a concentration of from about 0.01% to about 0.2%; and/or
ix) Tween 20 at a concentration of from about 0.0005% to about 0.01%.

18. The aqueous reaction mixture of any one of the preceding claims, wherein the reaction mixture comprises:
i) tetramethyl ammonium chloride at a concentration of from about 2.5 M to about 2.75 M;
ii) colloidal gold at a concentration of from about 1×10⁷ gold particles/μL to about 3×10⁷ gold particles/μL;
iii) formamide at a concentration of from about 15% to about 25%;
iv) dextran at a concentration of from about 1% to about 3%;
v) SSPE buffer at a concentration of from about 1% to about 3%;
vi) Denhardt's Solution at a concentration of from about 1× to about 3×;
vii) EDTA at a concentration of from about 1 mM to about 3 mM;
viii) sodium dodecyl sulfate at a concentration of from about 0.01% to about 0.1%; and/or
ix) Tween 20 at a concentration of from about 0.001% to about 0.004%.

19. The aqueous reaction mixture of any one of the preceding claims, wherein the aqueous reaction mixture is in a container, wherein the container further contains
(a) a first immiscible liquid, wherein the first immiscible liquid is less dense than the aqueous reaction mixture, and/or
(b) a second immiscible liquid, wherein the second immiscible liquid is more dense than the aqueous reaction mixture.

20. The aqueous reaction mixture of claim 19, wherein the container contains both a first and a second immiscible liquid.

21. A hybrid capture method for enrichment of target nucleic acid molecules from a nucleic acid sample containing target nucleic acid molecules and non-target nucleic acid molecules, the method comprising:
i) forming the aqueous reaction mixture of any one of claims 1-20;
ii) incubating the aqueous reaction mixture at a hybridization temperature for at least about 1 minute and less than about 1 hour to thereby hybridize at least a portion of the bait oligonucleotides to at least a portion of the target nucleic acid molecules, wherein the hybridization temperature is about 65° C.; and then
iii) immobilizing at least a portion of the bait oligonucleotides on one or more solid surfaces, thereby producing immobilized target nucleic acid molecule-bait oligonucleotide complexes;
iv) separating at least a portion of the non-target nucleic acid molecules from the immobilized target nucleic acid molecule-bait oligonucleotide complexes; and
v) recovering target nucleic acid molecules from the one or more solid surfaces, or amplification products thereof, thereby providing a polynucleotide mixture enriched at least 250-fold for target nucleic acid molecules relative to the nucleic acid sample or enriched at least 250-fold for a baited region above genomic background.

22. A hybrid capture method for enrichment of target nucleic acid molecules from a nucleic acid sample containing target nucleic acid molecules and non-target nucleic acid molecules, the method comprising:
i) forming the aqueous reaction mixture of any one of claims 1-20;
ii) incubating the aqueous reaction mixture at a hybridization temperature of about 65° C. for at least about 10 minutes to thereby hybridize at least a portion of the bait oligonucleotides to at least a portion of the target nucleic acid molecules and produce a plurality of target nucleic acid molecule-bait oligonucleotide complexes; and then
iii) immobilizing at least a portion of the bait oligonucleotides on one or more solid surfaces, thereby producing immobilized target nucleic acid molecule-bait oligonucleotide complexes;
iv) separating at least a portion of the non-target nucleic acid molecules from the immobilized target nucleic acid molecule-bait oligonucleotide complexes;
v) recovering target nucleic acid molecules from the one or more solid surfaces, or amplification products thereof, thereby forming an enriched polynucleotide mixture of target and non-target nucleic acid molecules, wherein the polynucleotide mixture is enriched relative to the nucleic acid sample; and
vi) sequencing at least a portion of the nucleic acids in the enriched polynucleotide mixture.

23. The method of claim 21 or 22, wherein an on-target rate of at least about 65% is achieved within a 10 minute incubation of the aqueous reaction mixture at the hybridization temperature.

24. The method of claim 21 or 22, wherein the method provides an enrichment of target nucleic acid molecules or baited region in the enriched polynucleotide mixture of at least 500-fold relative to a sample that is not enriched.

25. The method of claim 21 or 22, wherein target nucleic acid molecules of the enriched polynucleotide mixture comprise at least about 75% of total target and non-target nucleic acid molecules in the enriched polynucleotide mixture.

26. The method of claim 21, wherein forming the aqueous reaction mixture comprises:
i) forming a reaction pre-mixture comprising the nucleic acid sample, water, and bait oligonucleotides;
ii) forming a concentrated pre-mixture by reducing the volume of the reaction pre-mixture to a reduced volume, thereby increasing the concentration of target nucleic acid molecules, non-target nucleic acid molecules, and bait oligonucleotides, wherein the reduced volume is less than the volume of the reaction mixture; and
iii) contacting the concentrated pre-mixture with a volume of hybridization buffer, wherein the combined volumes of the hybridization buffer and the volume of the concentrated pre-mixture, if any, equal the volume of the aqueous reaction mixture, thereby forming a re-suspended pre-mixture having a volume equal to the volume of the aqueous reaction mixture; and
iv) denaturing the target and non-target nucleic acid molecules of the re-suspended pre-mixture by:
a) heating the re-suspended pre-mixture to a denaturing temperature; and then
b) cooling the re-suspended pre-mixture to a hybridization temperature, thereby forming the aqueous reaction mixture.

27. The method of claim 26, wherein reducing the volume of the reaction pre-mixture to a reduced volume comprises concentrating the pre-mixture to dryness.

28. The method of claim 26, wherein the denaturing temperature is at least about 90° C.-99° C., and the denaturing comprises incubating the nucleic acid sample at the denaturing temperature for at least about 5 minutes.

29. The method of any one of claims 21-28, wherein the separating comprises removing aqueous components of the reaction mixture from the immobilized target nucleic acid molecule-bait oligonucleotide complexes, thereby removing nucleic acids and blocking oligonucleotides that are not hybridized to the bait oligonucleotides, and then applying an aqueous wash buffer to the immobilized target nucleic acid molecule-bait oligonucleotide complexes.

30. The method of any one of claims 22-29, wherein the incubating the aqueous reaction mixture at the hybridization temperature comprises incubating the reaction mixture for at least about 30 minutes and less than about 240 minutes at about 65° C. to thereby hybridize at least a portion of the bait oligonucleotides to at least a portion of the target nucleic acids.

31. The method of any one of claims 22-30, wherein an on-target rate of at least about 75% is achieved within a 30 minute incubation of the aqueous reaction mixture at the hybridization temperature.

32. The method of any one of claims 22-31, wherein an on-target rate of at least about 80% is achieved within a 60-90 minute incubation of the aqueous reaction mixture at the hybridization temperature.

33. The method of any one of claims 21-29, wherein the incubating the aqueous reaction mixture at the hybridization temperature comprises incubating the reaction mixture for between about 10 minutes and 30 about minutes at about 65° C. to thereby hybridize at least a portion of the bait oligonucleotides to at least a portion of the target nucleic acids.

34. The method of any one of claims 21-33, wherein the step of immobilizing on one or more solid surfaced comprises contacting the bait oligonucleotides with beads comprising an affinity agent that specifically binds the label of the bait oligonucleotides.

35. The method of claim 34, wherein the immobilizing comprises contacting the bait oligonucleotides with beads comprising capture agent at a temperature of from about 37° C. to about 75° C. for at least about 10 minutes.

36. The method of claim 34, wherein the immobilizing comprises contacting the bait oligonucleotides with beads comprising capture agent at a temperature of from about 60° C. to about 70° C. for at least about 20 minutes.

37. The method of claim 36, wherein the label of the bait oligonucleotides comprises biotin and the capture agent comprises avidin or streptavidin.

38. The method of any one of claims 21-37, wherein recovering comprises amplifying the immobilized enriched target nucleic acid molecules to produce amplification products thereof, and collecting the amplification products.

39. A method of enriching a plurality of target nucleic acid molecules from a nucleic acid sample containing target nucleic acid molecules and non-target nucleic acid molecules, the method comprising:
i) incubating the sample containing target nucleic acid molecules and non-target nucleic acid molecules in an aqueous reaction mixture containing a plurality structurally distinct bait oligonucleotides at a hybridization temperature of about 65° C. for at least about 10 minutes to thereby hybridize at least a portion of the bait oligonucleotides to at least a portion of the target nucleic acid molecules, wherein:
a) the bait oligonucleotides comprise sequences complementary to the plurality of target nucleic acid molecules; and
b) the concentration of bait oligonucleotides in the aqueous reaction mixture is at least 0.5 pmol/μL; and
ii) immobilizing at least a portion of the bait oligonucleotides on one or more solid surfaces, thereby producing immobilized target nucleic acid molecule-bait oligonucleotide complexes;
iii) washing said one or more solid surfaces to provide one or more washed solid surfaces comprising immobilized target nucleic acid molecule-bait oligonucleotide complexes; and
iv) recovering target nucleic acid molecules, or amplification products thereof, from the one or more washed solid surfaces, thereby providing a polynucleotide mixture enriched for target nucleic acid molecules relative to the nucleic acid sample.

40. The method of claim 39, wherein the method enriches the polynucleotide mixture by at least 250-fold for target nucleic acid molecules or baited region relative to the nucleic acid sample within a 10 minute incubation of the aqueous reaction mixture at the hybridization temperature.

41. The method of claim 39, wherein the method further comprises sequencing at least a portion of the nucleic acids in the enriched polynucleotide mixture, wherein an on-target rate of at least about 65% is achieved within a 10 minute incubation of the aqueous reaction mixture at the hybridization temperature.

42. The method of claim 39, wherein the method further comprises sequencing at least a portion of the nucleic acids in the enriched polynucleotide mixture, wherein an on-target rate of at least about 75% is achieved within a 30 minute incubation of the aqueous reaction mixture at the hybridization temperature.

43. The method of claim 39, wherein the method further comprises sequencing at least a portion of the nucleic acids in the enriched polynucleotide mixture, wherein an on-target rate of at least about 80% is achieved within a 60-90 minute incubation of the aqueous reaction mixture at the hybridization temperature.

44. The method of any one of claims 39-43, wherein the concentration of bait oligonucleotides in the aqueous reaction mixture is at least 0.75 pmol/μL.

45. The method of any one of claims 39-43, wherein the concentration of bait oligonucleotides in the aqueous reaction mixture is from about 1 pmol/μL to about 2 pmol/μL.

46. The method of any one of claims 39-43, wherein the total concentration of target and non-target nucleic acids in the aqueous reaction mixture is from about 100 ng/μL to about 1,500 ng/μL.

47. The method of any one of claims 39-46, wherein the aqueous reaction mixture has a volume of from about 1 μL to 5 μL.

48. The method of any one of claims 39-46, wherein the aqueous reaction mixture has a volume of about 2 μL.

49. The method of any one of claims 39-48, wherein the method comprises incubating the sample containing target nucleic acid molecules and non-target nucleic acid molecules in the aqueous reaction mixture containing a plurality structurally distinct bait oligonucleotides at the hybridization temperature for at least about 30 minutes.

50. The method of any one of claims 39-48, wherein the method comprises incubating the sample containing target nucleic acid molecules and non-target nucleic acid molecules in the aqueous reaction mixture containing a plurality structurally distinct bait oligonucleotides at the hybridization temperature for at least about 10 minutes and less than about 4 hours.
